(19) United States
(12) Patent Application Publication
(10) Pub. No.: US 2024/0329050 A1
(43) Pub. Date: Oct. 3, 2024

(54) METHODS OF DETECTING PENTASACCHARIDES AND TREATMENT AND MONITORING OF LYSOSOMAL STORAGE DISEASE

(71) Applicant: Washington University, St. Louis, MO (US)

(72) Inventors: Xuntian JIANG, St. Louis, MO (US); Daniel ORY, St. Louis, MO (US)

(21) Appl. No.: 18/579,811

(22) PCT Filed: Jul. 18, 2022
(86) PCT No.: PCT/US22/73860

§ 371 Date: Jan. 16, 2024

Related U.S. Application Data
(60) Provisional application No. 63/222,524, filed on Jul. 16, 2021.

(51) Int. Cl. G01N 33/66; G01N 30/02; G01N 33/60

(52) U.S. Cl. CPC G01N 33/66; G01N 30/02; G01N 33/60; G01N 2030/027; G01N 2800/28; G01N 2800/52

(57) ABSTRACT

The present disclosure provides for biomarkers (pentasaccharides) for lysosomal storage disease (e.g., GM1-gangliosidosis). The biomarkers can improve diagnosis and assess disease severity and therapeutic efficacy. The methods include measuring pentasaccharid levels in a biological sample from a subject. Treatment of GM 1-gangliosidosis can include substrate reduction therapy or restoration or replacement of enzymatic activity.

### CROSS REFERENCE TO RELATED APPLICATIONS

This application claims priority of U.S. Provisional application No. 63/222,524, filed Jul. 16, 2021, the disclosure of which is hereby incorporated by reference in its entirety.

### GOVERNMENT SUPPORT

This invention was made with government support under NS114156 awarded by the National Institutes of Health. The government has certain rights in the invention.

### FIELD OF THE TECHNOLOGY

The present disclosure encompasses compositions and methods to quantify and analyze various pentasaccharides and the use thereof to measure pathological features and/or clinical symptoms of lysosomal storage disease (e.g. GM1-gangliosidosis).

### BACKGROUND

GM1-gangliosidosisis is a rare, progressive neurodegenerative disorder with an estimated incidence of between 1 in 100,000 and 1 in 200,000. This disorder is caused by mutations in the GLB1 encoding lysosomal β-galactosidase whose level of activity is inversely proportional to disease severity. The deficiency of β-galactosidase enzyme activity leads to accumulation of glycoconjugates with a terminal β-galactose, including gangliosides GM1 and GA1, oligosaccharides, glycopeptide, and keratan sulfate. The GM1-gangliosidosis affects the nervous system and numerous visceral organs. The clinical manifestations of GM1-gangliosidosis are heterogeneous and the disease is divided into three categories based on age of disease onset, which correlates to disease progression and severity of symptoms. Type I (infantile form) with enzyme activity of 0.07-1.3% of normal is the most aggressive form with onset of symptoms before 12 months and death by 4 years of age. The infants present with hepatosplenomegaly, cardiomyopathy, developmental regression, an exaggerated startle response, coarse facial features, skeletal dysplasia, seizures, cherry-red maculae, and severe CNS degeneration. Type II with enzyme activity in the order of 0.3-4.8% of normal includes late-infantile form with symptom onset at age one to two years and juvenile form with symptoms develop between 2 and 5 years of age. Type II patients demonstrate motor and cognitive regression, corneal clouding, ataxia, progressive bony changes, and seizures at slower progression. Type III (chronic or adult) with an enzyme activity in the region of 5-10% of normal is the mildest phenotype and characterized by symptom onset in young adulthood that includes dystonia, gait disturbance, muscle atrophy, dysarthria, corneal clouding, and cardiomyopathy. Over 100 mutations have been reported in GLB1 and there is no clear correlation between genotype and phenotype.

Currently, there are no effective therapies for the treatment of GM1-gangliosidosis. Two treatment strategies that are under development are substrate reduction therapy and restoration or replacement of enzymatic activity. One of major challenges for developing treatments for GM1-gangliosidosis is the difficulty in evaluation of treatment efficacy with clinical endpoints due to the small and heterogeneous patient population as well as slow progression in the non-infantile patients.

Thus, a need exists in the art for compositions and methods using validated biomarkers as outcome measures to predict clinical benefits, accelerate drug development and aid in disease detection.

### SUMMARY

In an aspect, the present disclosure encompasses methods for detecting GM1-gangliosidosis in a subject having or suspected of having symptoms of GM1-gangliosidosis, the method generally comprises (a) measuring pentasaccharide levels, in a urine, a blood sample or a CSF sample obtained from the subject; and (b) using the measurements of (a) to classify the subject as having GM1-gangliosidosis.

Another aspect of the present disclosure provides methods for detecting GM1-gangliosidosis severity in a subject having or suspected of having symptoms of GM1-gangliosidosis, the method generally comprises (a) measuring pentasaccharide levels, in a urine, a blood sample or a CSF sample obtained from the subject; and (b) using the measurements of (a) to classify the severity GM1-gangliosidosis.

In some embodiments, measuring pentasaccharide levels includes processing the urine, the blood sample or the CSF sample from the subject comprising the steps of adding an internal standard, depleting one or more protein(s) and derivatizing one or more pentasaccharides. In some embodiments, the internal standard is a stable isotope labeled pentasacchride. In some embodiments, the stable isotope labeled pentasacchride is d₆-H3N2b. In some embodiments, protein precipitation is used to deplete one or more protein(s). In some embodiments, the pentasacchride are derivatized with 2-aminobenzoic acid (2-AA). In some embodiments, measuring further comprises performing liquid chromatography-mass spectrometry with the sample comprising derivatized pentasaccharide and the internal standard to detect and measure the concentration of at least one pentasaccharide. In some embodiments, the measured pentasacchride is H3N2a and/or H3N2b.

In some embodiments, the measured pentasacchride level is compared to a reference value of a healthy control. In some embodiments, if the measured pentasacchride level is greater than the reference value the subject is classified as having GM1-gangliosidosis.

The present disclosure also provides the use of the measured pentasacchride level to select a therapeutic agent or a diagnostic agent for a subject.

In some embodiments, the method further comprises administering a pharmaceutical composition to a subject classified has having GM1-gangliosidosis. In some embodiments, the pharmaceutical composition comprises a substrate reduction therapy, bone marrow transplantation, pharmacological chaperone, enzyme replacement therapy, or gene therapy. In some embodiments, the enzyme replacement or gene therapy is providing a functional β-galactosidase enzyme.

In still another aspect, the present disclosure provides methods for treating a subject or monitoring the effectiveness of a therapeutic agent, the method generally comprises (a) providing a first biological sample obtained from a subject; (b) administering a pharmaceutical composition to the subject; (c) providing a second biological sample obtained from the subject sometime after administration of the pharmaceutical composition; and (d) measuring in each sample a pentasacchride level.

In some embodiments, the biological sample is a urine, a blood, or a CSF sample. In some embodiments, measuring pentasaccharide levels includes processing the urine, the blood sample or the CSF sample from the subject comprising the steps of adding an internal standard, depleting one or more protein(s) and derivatizing one or more pentasaccharides. In some embodiments, the internal standard is a stable isotope labeled pentasacchride. In some embodiments, the stable isotope labeled pentasacchride is d₆-H3N2b. In some embodiments, protein precipitation is used to deplete one or more protein(s). In some embodiments, the pentasacchride are derivatized with 2-aminobenzoic acid (2-AA). In some embodiments, measuring further comprises performing liquid chromatography-mass spectrometry with the sample comprising derivatized pentasaccharide and the internal standard to detect and measure the concentration of at least one pentasaccharide.

In some embodiments, the measured pentasacchride is H3N2a and/or H3N2b. In some embodiments, the measured pentasacchride level is compared to a reference value of a healthy control. In some embodiments, if a decrease in the pentasacchride level from the first sample relative to the second sample indicates the pharmaceutical composition is effective or no change or an increase in pentasacchride level indicates the pharmaceutical composition is ineffective. In some embodiments, the pharmaceutical composition comprises a substrate reduction therapy, bone marrow transplantation, pharmacological chaperone, enzyme replacement therapy, or gene therapy. In some embodiments, the enzyme replacement or gene therapy is providing a functional β-galactosidase enzyme. In some embodiments, the biological sample is a urine sample and the subject is classified as having GM1-gangliosidosis or treated for GM1-gangliosidosis when the measured level of H3N2b is greater than about 1.05 ng/μg creatinine. In some embodiments, the biological sample is a plasma sample and the subject is classified as having GM1-gangliosidosis or treated for GM1-gangliosidosis when the measured level of H3N2b is greater than about 8.9 ng/mL. In some embodiments, the biological sample is a CSF sample and the subject is classified as having GM1-gangliosidosis or treated for GM1-gangliosidosis when the measured level of H3N2b is greater than about 15.2 ng/mL.

Other aspects and iterations of the disclosure are described more thoroughly below.

### BRIEF DESCRIPTION OF THE FIGURES

The patent or patent application file contains at least one photograph executed in color. Copies of this patent or patent application publication with color photographs will be provided by the Office upon request and payment of the necessary fee.

FIG. 1A and FIG. 1B show ganglioside GM1 in human plasma and CSF. FIG. 1A shows Type II Ganglioside GM1 in human plasma samples. FIG. 1B shows type II Ganglioside GM1 in human CSF samples. Data are presented as median±interquartile peak area ratio of analyte to internal standard. GM1(16:0), C16:0 ganglioside GM1; GM1(18:0), C18:0 ganglioside GM1; GM1(20:0), C20:0 ganglioside GM1. The internal standard is d3-GM1(18:0). Mann-Whitney U-test was used to compare GM1 gangliosidosis and controls. ns: not significant; *: p<0.05

FIG. 2A and FIG. 2B shows separation of H3N2a and H3N2b. FIG. 2A shows chromatograms of 2-AA derivatized H3N2a and H3N2b in type II GM1 gangliosidosis human urine, plasma, CSF, and cat brain stem samples. H3N2a is not found in the cat brain stem. FIG. 2B shows enhanced product ion spectra of [M+H]+ ions of 2-AA derivatized H3N2a, H3N2b, and 2 interfering peaks (peaks 1 and 3) show that they are isomeric pentasaccharides.

FIG. 3A-FIG. 3D show pentasaccharide biomarkers H3N2a and H3N2b in human. FIG. 3A show H3N2a in human urine, plasma, and CSF samples. Data are presented as median±interquartile area on log scale. Mann-Whitney U-test was used to compare GM1 gangliosidosis and controls. ****: p<0.0001. FIG. 3B show H3N2b in human urine, plasma, and CSF samples. Data are presented as median±interquartile peak area on log scale. Mann-Whitney U-test was used to compare GM1 gangliosidosis and controls. ****: p<0.0001. FIG. 3C show relations between urine, plasma, and CSF H3N2a and H3N2b and age/disease type. FIG. 3D show comparison of H3N2a and H3N2b in late infantile and juvenile GM1 gangliosidosis patients. Data are presented as median±interquartile peak area. Mann-Whitney U-test was used to compare late infantile and juvenile GM1 gangliosidosis patients.*: p<0.05; **: p<0.01.

FIG. 4A and FIG. 4B show pentasaccharide biomarker H3N2b in cat. FIG. 4A shows H3N2b in cat CNS samples. Data are presented as mean±standard deviation peak area. Comparison of normal, GM1 gangliosidosis treated with AAV gene therapy, and untreated GM1 gangliosidosis was performed with the one-way ANOVA and Tukey's multiple comparisons as post hoc test. ns: not significant; *: p<0.05; **: p<0.01; ***: p<0.001; ****: p<0.0001. FIG. 4B shows correlation of H3N2b in cat CNS with □-galactosidase activity. H3N2b level and β-galactosidase activity were fitted with nonlinear one phase decay least squares fit, and Spearman rank based correlation coefficients indicate that they are negatively correlated. ****: p<0.0001.

FIG. 5A-FIG. 5C show synthesis of H3N2b and its internal standard and confirmation of H3N2b structure. FIG. 5A shows synthesis of H3N2b and d6-H3N2b. FIG. 5B shows chromatogram (detected by product ion scan) and product ion spectra of [M+H]+ ion of 2-AA derivatized synthetic H3N2b after purification with HPLC. FIG. 5C shows chromatogram (detected by product ion scan) and product ion spectra of [M+H]+ ion of 2-AA derivatized H3N2b in GM1 gangliosidosis urine.

FIG. 6A-FIG. 6F show establishment of normal cutoffs for urine, plasma, and CSF H3N2b. FIG. 6A shows H3N2b normalized to creatinine in human urine samples. H3N2b concentrations are presented as median±interquartile on log scale. Mann-Whitney U-test was used to compare GM1 gangliosidosis and controls. ***: p<0.001. FIG. 6B shows H3N2b concentrations in human plasma samples. The measured H3N2b concentrations that were below the LLOQ (1 ng/mL) were used for purpose of plotting, though the % CV and % RE for these samples may not meet acceptance criteria for the validated assay. H3N2b concentrations are presented as median±interquartile on log scale. Mann-Whitney U-test was used to compare GM1 gangliosidosis and controls. ****: p<0.0001. FIG. 6C shows H3N2b concentrations in human CSF samples. The measured H3N2b concentrations that were below the LLOQ (1 ng/mL) were used for purpose of plotting, though the % CV and % RE for these samples may not meet acceptance criteria for the validated assay. H3N2b concentrations are presented as median±interquartile on log scale. Mann-Whitney U-test was used to compare GM1 gangliosidosis and controls. **: p<0.01. FIG. 6D shows determination of cutoff for urine using ROC curve. FIG. 6E shows determination of cutoff for plasma using ROC curve. FIG. 6F shows determination of cutoff for CSF using ROC curve.

FIG. 7A-FIG. 7C show response of H3N2b in urine FIG. 7A, plasma FIG. 7B, and CSF FIG. 7C to intravenous AAV9 gene therapy in the participants of Phase 1/2 clinical trial. The participants IVGT001, IVGT003, IVGT004, IVGT006, IVGT007, IVGT008 received low dose of gene therapy, and high dose was administered to IVGT010, IVGT011, and IVGT012. The post-treatment CSF samples were not available from IVGT012. Lines indicate longitudinal data. The green dash-dotted lines show the cutoffs for normal ranges as derived from ROC curve analysis. Simple paired sample t-test was used to compare each follow up month versus baseline (month 0) for H3N2b. Significant difference compared to pretreatment was found for urine H3N2b at 1 (p<0.01), 3 (p<0.01), 6 (p<0.01), and 12 (p<0.05) months, for plasma H3N2b at 1 (p<0.05), 3 (p<0.05), 6 (p<0.01), 12 (p<0.01), and 18 (p<0.05) months, and for CSF H3N2b at 3 (p<0.01), 6 (p<0.001), and 12 (p<0.05) months. An integrative LME for repeated measures across all the time points was conducted, the overall differences across time were tested by a Type III F test, and the differences between the least square means estimated from the LME between two time points were derived and tested against month 0 with Tukey-Kramer adjusted p values. The overall Type III tests for the time effect on urine, plasma, and CSF H3N2b were significant with p values <0.0001, 0.0002, and <0.0001, respectively.

FIG. 8A-FIG. 8D show structural identification of H3N2a and H3N2b. FIG. 8A shows fragmentation of [M+Na]+ ions of 2-AA derivatized H3N2a and H3N2b. FIG. 8B shows fragmentation of [M−H]− ions of NaIO4-NaBH4 treated 2-AA derivatized H3N2a and H3N2b. FIG. 8C shows fragmentation of [M+H]+ ions of NaIO₄—NaBH₄ treated 2-AA derivatized H3N2a and H3N2b. FIG. 8D show stepwise digestion of H3N2a and H3N2b with β1,4 galactosidase, β-N-acetylglucosaminidase, α-1-2,3,6-mannosidase, and β-mannosidase.

### DETAILED DESCRIPTION

The present disclosure is based, at least in part, on the discovery of pentasaccharides as biomarkers for improving diagnosis and assessing disease severity and therapeutic efficacy.

GM1-gangliosidosis is a rare, progressive neurodegenerative disorder with an estimated incidence of between 1 in 100,000 and 1 in 200,000. This disorder is caused by mutations in the GLB1 gene encoding β-galactosidase whose level of activity is inversely proportional to disease severity. The enzyme analysis of the β-galactosidase is used as first line diagnosis; however, false negatives were reported due to the use of artificial substrates in the in vitro enzyme assay. A low cost and improved diagnostic assay is needed. Currently there are no effective therapies for the treatment of GM1-gangliosidosis. Two treatment strategies are described: 1) substrate reduction therapy; and 2) restoration or replacement of enzymatic activity. Thus, treatment of GM1-gangliosidosis can include substrate reduction therapy or restoration or replacement of enzymatic activity. A major challenge in development of new drugs is difficulty in evaluation of treatment efficacy with clinical endpoints due to the small and heterogeneous patient population as well as slow progression in the non-infantile patients. Markers to predict clinical benefits is critical to accelerate drug development, particularly in rare diseases.

The present disclosure identifies 2 pentasaccharides that are significantly elevated in patient biological samples (e.g., plasma, urine, CSF). As described herein, the discovered pentasaccharides are referred as to H3N2a and H3N2b, where H is hexose, and N is N-acetylhexosamine. In addition, the present disclosure found that the H3N2b but not H3N2a was detectable and elevated in GM1-gangliosidosis cat central nervous system (CNS) samples including brain stem, cerebellum, cervical intumescence, frontal cortex, lumbar intumescence, occipital cortex, parietal cortex, temporal cortex, and thalamus. It was further discovered that CNS H3N2b levels were decreased in response to adeno-associated viral (AVV) gene therapy treatment.

The H3N2a and H3N2b are natural substrates of β-galactosidase that play a central role in GM1-gangliosidosis; thus, its level is inversely proportional to enzyme activity and directly proportional to the disease severity and progression. Unlike other GM1-gangliosidosis biomarkers that can only serve as either peripheral or CNS biomarker, H3Na and H3N2b can assess the disease progression and treatment efficacy in both CNS and visceral organs. In addition, they provide large dynamic range to evaluate the disease progression and treatment efficacy. Their potential to correlate with disease severity and progression and easy to measure serially in biological samples such as biofluids (e.g., urine, plasma, CSF) make them appropriate as prognostic and monitoring biomarker. The pentasaccharide's sensitive response to AAV treatment indicates that they are pharmacodynamics/response biomarkers for treatment targeted at β-galactosidase activity.

The plasma and urine H3N2a and H3N2b can be used as diagnostic biomarker for GM1-gangliosidosis. The H3N2a and H3N2b level will be measured in plasma or urine from the patients with one or more of the following indications: positive family history for GM1-gangliosidosis, neurodegenerative symptoms, skeletal symptoms, or cherry red spot. The β-galactosidase activity assay and genetic testing of the GLB1 gene will be ordered for those with H3N2b above the cutoff. Applying H3N2a and H3N2b together with the enzymatic assay and genetic testing in the diagnosis would provide more accurate diagnosis for this disorder.

H3N2a and H3N2b in CSF, plasma, or urine can be used as prognostic and monitoring biomarker to monitor the disease progression. They can be used in GM1-gangliosidosis to stratify subgroups in whom the disease progresses at different rates. This could facilitate our understanding of the pathogenesis and allow us to differentiate phenotypes within a heterogeneous GM1-gangliosidosis population. Ultimately, such findings help facilitate the development of treatment for GM1-gangliosidosis.

The H3N2a and H3N2b in CSF, plasma, or urine can be served as pharmacodynamics/response biomarker in the development of treatment for GM1-gangliosidosis that restores or replaces enzymatic activity, including bone marrow transplantation, pharmacological chaperone, enzyme replacement therapy, and gene therapy. The pentasaccharide biomarkers can provide information about: 1) whether the enzymatic activity is increased after treatment; 2) to what degree the enzymatic activity is increased; 3) whether the enzymatic activity is increased in peripheral organs or CNS; and/or 4) selection of optimal dosing and administration route.

Disclosed herein, the oligosaccharides in GM1-gangliosidosis patient samples were profiled and identified 2 pentasaccharides that are significantly elevated in patient plasma, urine, and CSF. The pentasaccharides are referred as to H3N2a and H3N2b, where H is hexose, and N is N-acetylhexosamine. It was further found that the H3N2b but not H3N2a was detectable and elevated in GM1-gangliosidosis cat central nervous system (CNS) samples including brain stem, cerebellum, cervical intumescence, frontal cortex, lumbar intumescence, occipital cortex, parietal cortex, temporal cortex, and/or thalamus. It was further discovered that CNS H32b levels were decreased in response to adeno-associated viral (AVV) gene therapy treatment.

The diagnostic technologies using H3N2a and H3N2b can be used in clinical laboratories. Currently, enzyme analysis of the β-galactosidase and molecular genetic testing of the GLB1 gene are used to diagnose GM1-gangliosidosis, and the enzyme analysis is used as first line diagnosis, because of cost and difficulty in interpreting unclear results in the molecular genetic testing. However, there were reports that β-galactosidase testing led to false negatives because the use of artificial substrates in the in vitro enzyme assay may not exactly replicate in vivo enzyme activity with natural substrates. H3N2a and H3N2b in urine were used for diagnosis of GM1-gangllosidosis, but the assays are semi-quantitative due to lack of authentic standards and ill-defined structures, and no reference ranges were established. An improved diagnostic assay is needed for unambiguous identification of GM1-gangliosidosis patients at low cost. Described herein are methods to develop an FDA compliant assay for H3N2b, and establish reference ranges for GM1-gangliosidosls and cutoff for diagnosis. As such, clinical laboratories can implement the H3N2b assay with improved diagnostic accuracy.

Applicants have discovered that certain methods to quantify pentasaccharides (e.g., H3N2a and H3N2b) which can be used to track the GM1-gangliosidosis process across various disease stages and treatment regimens. Given the extremely large variability in oligosaccharides, abundance and detectability, the use of pentasaccharides to stage subjects having or suspected of having symptoms associated with GM1-gangliosidosis and guide treatment decisions has been elusive. However, Applicant has identified methods of quantifying specific pentasaccharides which are particularly useful for identifying disease prevalence, disease progression and the development of certain pathophysiological changes, in addition to guiding treatment decisions.

The methods disclosed herein employ unique combinations of processing steps and internal standards which transform a biological sample into a sample suitable for quantifying various pentasaccharides. For instance, in some methods of the present disclosure, the processing steps deplete certain proteins while derivatizing pentasaccharides. In other methods of the present disclosure, the processing steps deplete certain proteins while enriching for derivatized pentasaccharides. Certain methods disclosed herein are particularly suited for quantifying H3N2b. Also described herein are uses of H3N2b to measure clinical signs and symptoms of GM1-gangliosidosis, diagnose GM1-gangliosidosis, and direct treatment of GM1-gangliosidosis. These and other aspects and iterations of the invention are described more thoroughly below.

### I. Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

As used herein, the term “subject” refers to a mammal, preferably a human. The mammals include, but are not limited to, humans, primates, livestock, rodents, and pets. A subject may be waiting for medical care or treatment, may be under medical care or treatment, or may have received medical care or treatment.

As used herein, the term “control population,” “normal population” or a sample from a “healthy” subject refers to a subject, or group of subjects, who are clinically determined to not have a tauopathy or Aβ amyloidosis, or a clinical disease associated with Aβ amyloidosis (including but not limited to Alzheimer's disease), based on qualitative or quantitative test results. A “normal” subject is usually about the same age as the individual to be evaluated, including, but not limited to, subjects of the same age and subjects within a range of 5 to 10 years.

The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented. Accordingly, a subject in need of treatment may or may not have any symptoms or clinical signs of disease.

### II. Methods for Measuring Pentasaccharides

The present disclosure provides methods for measuring pentasaccharides (e.g. H3N2a and H3N2b) in a biological sample by mass spectrometry. Generally speaking, methods of the present disclosure for measuring pentasaccharides in a biological sample comprise providing a biological sample, processing the biological sample by depleting one or more protein and then derivatizing one or more pentasaccharides, and then optionally concentrating the derivatized product, and performing liquid chromatography-mass spectrometry with the sample comprising derivatized pentasaccharides and an internal standard (e.g. d₆-H3N2b) to detect and measure the concentration (relative or absolute) of at least one derivatized pentasaccharides. Thus, in practice, the disclosed methods use at least one derivatized pentasaccharides to detect and measure the amount of pentasaccharides present in the biological sample.

In one example, a method of the present disclosure comprises (a) providing a biological sample selected from a blood sample, a urine sample or a CSF sample; (b) removing proteins from the biological sample by protein precipitation and separating the precipitated proteins to obtain a supernatant; (c) derivatizing one or more pentasaccharides from the supernatant and then optionally concentrating the resultant derivatized product; and (d) performing liquid chromatography-mass spectrometry with the sample comprising derivatized pentasaccharides to detect and measure the concentration of at least one pentasaccharide.

In another example, a method of the present disclosure comprises (a) providing a biological sample selected from a blood sample, a urine sample or a CSF sample; (b) adding an internal standard to the sample; (c) removing proteins from the biological sample by protein precipitation and separating the precipitated proteins to obtain a supernatant; (d) derivatizing one or more pentasaccharides from the supernatant and then optionally concentrating the resultant derivatized product; and (e) performing liquid chromatography-mass spectrometry with the sample comprising derivatized pentasaccharides to detect and measure the concentration of at least one pentasaccharide.

The present disclosure is not limited to any one particular method to quantitatively assess pentasaccharide levels. In some embodiments, the approaches use internal normalization for comparing relative concentration of at least one pentasaccharide. Other methods known in the art may also be used. When using an internal synthetic labeled standard for absolute quantification, the labeled standard is preferably spiked into the sample prior to processing the sample. In certain embodiments, the internal standard is d₆-H3N2b.

In an exemplary embodiment, pentasaccharide levels is measured by high-resolution mass spectrometry. Suitable types of mass spectrometers are known in the art. These include, but are not limited to, quadrupole, time-of-flight, ion trap and Orbitrap, as well as hybrid mass spectrometers that combine different types of mass analyzers into one architecture (e.g., Orbitrap Fusion™ Tribrid™ Mass Spectrometer from ThermoFisher Scientific). Following one or more clean-up steps, pentasaccharide may be separated by a liquid chromatography system interfaced with a high-resolution mass spectrometer. The chromatography system may be optimized by routine experimentation to produce a desired LC-MS pattern. A wide array of LC-MS techniques may be used to quantitatively analysis pentasaccharide. Non-limiting examples include selected-reaction monitoring, parallel-reaction monitoring, selected-ion monitoring, and data-independent acquisition. In an exemplary embodiment, a mass spectrometry protocol outlined in the Examples is used.

The biological sample, suitable internal standards, and the steps of depleting one or more protein, purifying pentasaccharide, derivatizing pentasaccharide, and mass spectrometry are described in more detail below.

### (a) Biological Sample

Suitable biological samples include a urine sample, a blood sample or a cerebrospinal fluid (CSF) sample obtained from a subject. In some embodiments, the subject is a human. A human subject may be waiting for medical care or treatment, may be under medical care or treatment, or may have received medical care or treatment. In various embodiments, a human subject may be a healthy subject, a subject at risk of developing GM1-gangliosidosis, a subject with signs and/or symptoms of GM1-gangliosidosis, or a subject diagnosed with GM1-gangliosidosis. In other embodiments, the subject is a laboratory animal. In a further embodiment, the subject is a laboratory animal genetically engineered to express a mutated and or deficient β-galactosidase.

CSF may have been obtained by lumbar puncture with or without an indwelling CSF catheter. Multiple blood or CSF samples contemporaneously collected from the subject may be pooled. Blood may have been collected by veni-puncture with or without an intravenous catheter, or by a finger stick (or the equivalent thereof). Once collected, blood or CSF samples may have been processed according to methods known in the art (e.g., centrifugation to remove whole cells and cellular debris; use of additives designed to stabilize and preserve the specimen prior to analytical testing; etc.). Blood or CSF samples may be used immediately or may be frozen and stored indefinitely. Prior to use in the methods disclosed herein, the biological sample may also have been modified, if needed or desired, to include protease inhibitors, isotope labeled internal standards, detergent(s) and chaotropic agent(s), and/or to deplete other analytes (e.g. proteins peptides, metabolites). Urine samples can be collected using methods and standards known in the art.

The size of the sample used can and will vary depending upon the sample type, the health status of the subject from whom the sample was obtained, and the analytes to be analyzed. CSF samples volumes may be about 0.01 mL to about 5 mL, or about 0.05 mL to about 5 mL. In a specific example, the size of the sample may be about 0.05 mL to about 1 mL CSF. Plasma sample volumes may be about 0.01 mL to about 20 mL.

### (b) Isotope-Labeled, Internal Pentasaccharide Standard

Isotope-labeled pentasaccharide (e.g. d₆-H3N2B) may be used as an internal standard to account for variability throughout sample processing and optionally to calculate an absolute concentration. Generally, an isotope-labeled, internal pentasaccharide standard is added before significant sample processing, and it can be added more than once if needed.

Isotope-labeled internal standards are described herein. As disclosed herein the present disclosure provides methods for synthesizing H3N2B and d₆-H3N2B. Accordingly, the present disclosure is also directed to compositions and methods comprising synthetic H3N2B and/or d₆-H3N2B.

Many general references providing commonly known chemical synthetic schemes and conditions useful for synthesizing the disclosed internal standards are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Internal standards as described herein can be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject standards, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry,” Plenum Press, London and New York 1973, in T W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/I, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art.

The standards disclosed herein can be made using procedures familiar to the person of ordinary skill in the art and as described herein. For example, synthetic H3N2B and/or d₆-H3N2B can be prepared according to the Scheme, general procedures (below), and/or analogous synthetic procedures. One of skill in the art can adapt the reaction sequences of the Schemes, general procedures, and Examples to fit the desired target molecule. Of course, in certain situations one of skill in the art will use different reagents to affect one or more of the individual steps or to use protected versions of certain of the substituents. Additionally, one skilled in the art would recognize that compounds of the disclosure can be synthesized using different routes altogether.

### (i) General Procedures

Representative synthetic procedures for the preparation of compounds of the invention are outlined below in the Scheme.

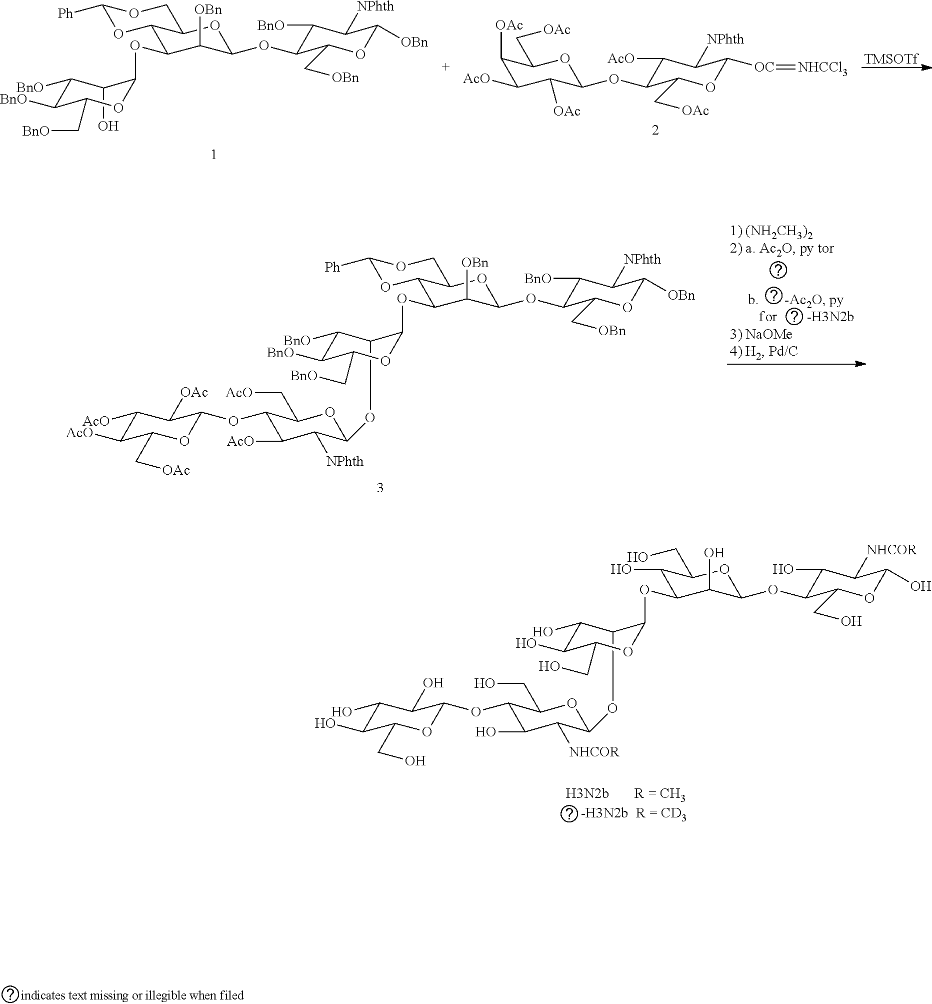

In an exemplary embodiment, the synthesis scheme includes the following steps: Benzyl-3,4,6-O-tri-benzyl-α-D-mannopyranosyl-(1→3)-2-O-benzyl-4,6-O-benzylidene-β-D-mannopyranosyl-(1→4)-3,6-O-di-benzyl-2-deoxy-phthalimido-β-D-glucopyranoside (2)—Compound 1 (10.68 g, 7.66 mmol, 1 eq) was dissolved in methanol/dichloromethane solvent mixture (3:2, 76 mL) followed by dropwise addition of sodium methoxide in methanol (0.5 M, 1.5 mL, 0.766 mmol, 0.1 eq). The reaction mixture was stirred overnight, quenched with ammonium chloride (0.65 g), sonicated for 5 min, and concentrated. The residue was purified by chromatography on a silica gel column (hexanes/ethyl acetate, 4:1→3:1→2:1→1:1) to give 2 (9.18 g, 88.5%). ESI-MS: [M+NH₄]⁺, calculated for C₈₂H₈₅N₂O₁₇⁺: m/z 1369.5843; found: m/z 1369.5812. ¹H NMR (CDCl₃, 400 MHz): δ 7.71-7.74 (m, 2H), 7.56-7.61 (m, 4H), 7.33-7.51 (m, 28H), 7.15-7.24 (m, 5H), 7.06-7.08 (m, 2H), 6.96-7.00 (m, 3H), 5.78 (brs, 1H), 5.65 (s, 1H), 5.49 (d, J=0.8 Hz, 1H), 5.29 (d, J=7.8 Hz, 1H), 4.92-5.06 (m, 5H), 4.84 (d, J=11.2 Hz, 1H), 4.80 (d, J=11.9 Hz, 1H), 4.77 (d, J=11.8 Hz, 1H), 4.75 (d, J=8.9 Hz, 1H), 4.51-4.67 (m, 6H), 4.41 (t, J=4.4 Hz, 2H), 4.33 (dd, J=10.4, 4.7 Hz, 1H), 3.88-4.24 (m, 8H), 3.80 (t, J=10.5 Hz, 2H), 3.58-3.69 (m, 5H); ¹³C NMR (CDCl₃, 100 MHz): δ 167.9, 138.9, 138.7, 138.5, 138.3, 138.0, 137.9, 137.5, 137.3, 133.8, 131.8, 128.9, 128.8, 128.6, 128.5, 128.4, 128.3, 128.2, 128.1, 128.0, 127.9, 127.8, 127.1, 126.2, 123.3, 101.7, 101.2, 98.9, 97.6, 79.1, 78.8, 78.4, 78.1, 77.6, 75.7, 75.5, 75.2, 75.0, 74.6, 74.4, 73.7, 73.6, 72.5, 71.7, 70.9, 69.3, 68.5, 68.3, 67.1, 55.9.

Benzyl-O-(2,3,4,6-tetra-O-acetyl-β-D-galactopyranosyl)-(1→4)-O-(3,6-di-O-acetyl-2-deoxy-2-phthalimido-β-D-glucopyranosyl)-(1→2)-O-(3,4,6-tri-O-benzyl-α-D-mannopyranosyl)-(1→3)-O-(6-O-acetyl-2,4-di-O-benzyl-β-D-mannopyranosyl)-(1→4)-3,6-O-di-benzyl-2-deoxy-phthalimido-β-D-glucopyranoside (4)—A mixture of compound 2 (8.1 g, 6 mmol, 1 eq) and lactosamine bromide 3 (19.2 g, 24 mmol, 4 eq) was azeotropically dried with toluene (2×100 mL), dissolve in dichoromethane (40 mL) and hexane (100 mL), evaporated, and then on high vacuum for 0.5 h. Freshly activated 4 Å molecular sieves (40 g) and dichoromethane (52 mL) were added, and the mixture was stirred for 0.5 hour under a nitrogen atmosphere in the dark. The mixture was cooled to −60° C., and 2,6-lutidine (2.79 mL, 24 mmol, 4 eq) was added. Silver trifluoromethanesulfonate (6.17 g, 24 mmol, 4 eq) was azeotropically dried with toluene (2×100 mL), then on high vacuum for 2 h, and dissolved in toluene (52 mL). The silver trifluoromethanesulfonate in toluene was added dropwise over 20 min to the mixture of compounds 2 and 3 and molecular sieves, and the final mixture was stirred and allowed to warm up to room temperature overnight. The reaction was quenched with saturated sodium thiosulfate solution. The mixture was diluted with dichloromethane and filtered through Celite. The organic phase is dried with magnesium sulfate, filtered, and concentrated in vacuum. The residue was purified by chromatography on a silica gel column (hexanes/ethyl acetate, 4:1→2:1→1:1) to give 4 (12.79 g, 92%). ESI-MS: [M+NH₄]⁺, calculated for C₁₁₄H₁₂₀N₃O₃₄⁺, m/z 2074.7748; found: m/z 2074.7707. ¹H NMR (CDCl₃, 400 MHz): δ 7.70-7.73 (m, 2H), 7.46-7.67 (m, 11H), 7.22-7.41 (m, 20H), 7.12-7.21 (m, 5H), 7.01-7.12 (m, 5H), 6.91-6.98 (m, 2H), 6.82-6.92 (m, 3H), 5.66 (t, J=9.6 Hz, 1H), 5.46 (s, 1H), 5.41 (s, 1H), 5.24-5.32 (m, 1H), 4.99-5.23 (m, 4H), 4.65-4.97 (m, 7H), 4.41-4.61 (m, 5H), 4.21-4.41 (m, 5H), 3.74-4.21 (m, 14H), 3.36-3.72 (m, 9H), 2.78 (m, 2H), 2.17 (s, 3H), 2.15 (s, 3H), 2.08 (s, 3H), 2.02 (s, 3H), 1.97 (s, 3H); ¹³C NMR (CDCl₃, 100 MHz): δ 169.9, 169.8, 169.7, 169.6, 169.4, 168.7, 167.5, 167.1, 138.3, 138.2, 137.9, 137.8, 137.3, 137.1, 131.4, 131.1, 131.0, 130.5, 128.8, 128.6, 128.5, 128.3, 128.9, 127.9, 127.8, 127.7, 127.6, 127.5, 127.5, 127.4, 127.3, 127.2, 127.2, 127.1, 126.5, 124.9, 123.5, 122.8, 101.8, 100.8, 100.4, 96.9, 94.7, 78.4, 77.7, 77.5, 76.3, 76.0, 74.5, 74.4, 74.1, 74.0, 73.5, 73.0, 72.4, 71.5, 71.3, 71.2, 70.8, 70.3, 70.1, 70.0, 69.9, 68.8, 68.2, 67.6, 66.3, 65.9, 60.9, 60.4, 55.2, 54.1, 21.1, 21.0, 20.2, 20.2, 20.1, 20.0.

O-β-D-galactopyranosyl-(1→4)-O-(2-acetamido-2-deoxy-β-D-glucopyranosyl)-(1→2)-O-α-D-mannopyranosyl-(1→3)-O-β-D-mannopyranosyl-(1→4)-2-acetamido-2-deoxy-α,β-D-glucopyranose (H3N2b)—A mixture of 4 (7.63 g, 3.71 mmol), n-butanol (26 mL), and ethylenediamine (13 mL, 196 mmol, 53 eq) was heated at 90° C. for overnight. The mixture was evaporated in vacuum, and the residue was dissolved in methanol (15 mL) and co-evaporated with toluene (3×100 mL), and then on high vacuum for 2 days. The residue was then dissolved in pyridine (19.6 mL, 244 mmol, 66 eq) and acetic anhydride (19.6 mL, 196 mmol, 53 eq) was added and the mixture stirred overnight. The reaction was then concentrated under reduced pressure and the residue co-evaporated with toluene (2×10 mL) and dissolved in tetrahydrofuran (30 mL). Liquid ammonia (300 mL) was condensed at −78° C. into a 3-neck flask (1000 mL) with solid sodium (5 g, 219 mmol, 59 eq) equipped with mechanic stir and Dewar-type condenser. The resulting deep blue solution was stirred for 30 min. A solution of product from last step in tetrahydrofuran was added, and the reaction mixture was stirred for an additional 2 hours at −78° C. The reaction was quenched with methanol (50 mL). The reaction vessel was subsequently removed from its cooling bath and warmed to 25° C., and the ammonia was evaporated overnight. The mixture was dissolved in water (100 mL) and neutralized with 12N hydrochloride. The organic solvent in the mixture was removed by rotavapor. The aqueous solution of crude H3N2b was washed with tetrahydrofuran-ethyl acetate (2:1) (2×150 mL) and desalted by chromatography on a short charcoal column (water/acetonitrile, 1:0→3:2). The pure H3N2b (0.96 g, 28.4% overall yield over 3 steps) that is an anomeric mixture was obtained as white solid by isolation on a Hypercarb porous graphitic carbon HPLC column (4.6×150 mm, 3 μm) (Thermo Scientific, Waltham, MA) protected with a SecurityGuard C18 guard column (4×3 mm). The mobile phases consisted of 0.1% acetic acid in water (solvent A) and acetonitrile (solvent B), and the flow rate was 1 mL/min. The gradient was as follows: 0 to 15 min, 5 to 8% solvent B; 15 to 15.1 min, 8 to 10% solvent B; 15.1 to 18 min, 10% solvent B; 18 to 18.1 min, 10 to 95% solvent B; 18.1 to 21 min, 95% solvent B; 21 to 21.1 min, 95 to 5% solvent B; 21.1 to 25 min, 5% solvent B. ESI-MS: [M+H]⁺, calculated for C₃₄H₅₉N₂O₂₆⁺, m/z 911.3351; found: m/z 911.3327. [M+Na]⁺, calculated for C₃₄H₅₈N₂O₂₆Na⁺, m/z 933.3170; found: m/z 933.3145. ¹H NMR (D₂O, 600 MHz) β 5.20 (d, J=3 Hz, 0.64H), 5.13 (s, 1H), 4.71-4.75 (m, 1H), 4.58 (d, J=7.2 Hz, 1H), 4.47 (d, J=7.8 Hz, 1H), 4.25 (dd, J=3 Hz, 6.6 Hz, 1H), 4.20 (s, br, 1H), 3.98 (d, J=10.8 Hz, 1H), 3.89-4.96 (m, 6H), 3.83-4.96 (m, 2H), 3.46-3.82 (m, 20H), 2.06 (s, 3H), 2.05 (s, 3H). ¹³C NMR (D₂O, 150 MHz): 177.3, 177.0, 105.5, 102.6, 102.1, 102.1, 97.5, 93.1, 83.0, 82.0, 81.6, 81.1, 79.0, 78.7, 77.9, 77.3, 77.2, 76.1, 75.1, 74.9, 74.5, 73.5, 72.9, 72.9, 72.7, 72.0, 71.8, 71.1, 69.9, 68.5, 64.3, 63.6, 63.4, 62.7, 62.6, 62.6, 58.7, 57.4, 56.2, 24.9, 24.8, 24.5.

O-β-D-galactopyranosyl-(1→4)-O-(2-d₃-acetamido-2-deoxy-β-D-glucopyranosyl)-(1→2)-O-α-D-mannopyranosyl-(1→3)-O-β-D-mannopyranosyl-(1→4)-2-d₃-acetamido-2-deoxy-α,β-D-glucopyranose (d₆-H3N2b)—The d₆-H3N2b (0.27 g, 20.4% overall yield over 3 steps) that is an anomeric mixture was prepared as white solid from 4 (2.96 g, 1.44 mmol) by reactions with: 1) n-butanol (10 mL), and ethylenediamine (5.1 mL, 76.3 mmol, 53 eq); 2) pyridine (7.6 mL, 95 mmol, 66 eq) and d₆-acetic anhydride (7.6 mL, 76.3 mmol, 53 eq); 3) liquid ammonia (120 mL) and sodium (1.95 g, 85 mmol, 59 eq) according to the procedures that was described for the preparation of H3N2b. ESI-MS: [M+H]⁺, calculated for C₃₄H₅₃D₆N₂O₂₆⁺, m/z 917.3727; found: m/z 917.3751. [M+Na]⁺, calculated for C₃₄H₅₂D₆N₂O₂₆Na⁺, m/z 939.3547; found: m/z 939.3572. ¹H NMR (D₂O, 600 MHz) δ 5.20 (d, J=3 Hz, 0.64H), 5.13 (s, 1H), 4.71-4.75 (m, 1H), 4.57 (d, J=7.2 Hz, 1H), 4.46 (d, J=7.8 Hz, 1H), 4.24 (dd, J=3 Hz, 6.6 Hz, 1H), 4.19 (s, br, 1H), 3.97 (d, J=10.8 Hz, 1H), 3.89-4.96 (m, 6H), 3.83-4.96 (m, 2H), 3.46-3.82 (m, 20H). ¹³C NMR (D₂O, 150 MHz) β 177.3, 177.1, 105.5, 102.6, 102.1, 102.0, 97.5, 93.1, 83.0, 82.0, 81.6, 81.0, 79.0, 78.7, 77.9, 77.3, 77.2, 76.1, 75.6, 75.1, 74.9, 74.5, 73.5, 72.9, 72.6, 72.0, 71.8, 71.1, 69.9, 68.5, 64.3, 63.6, 63.4, 62.7, 62.6, 62.5, 58.6, 57.4, 56.2.

### (c) Depleting One or More Protein

Methods of the present disclosure comprise a step wherein one or more protein is depleted from a sample. The term “deplete” means to diminish in quantity or number. Accordingly, a sample depleted of a protein may have any amount of the protein that is measurably less than the amount in the original sample, including no amount of the protein.

Protein(s) may be depleted from a sample by a method that specifically targets one or more protein, for example by affinity depletion, solid phase extraction, or other method known in the art. Targeted depletion of a protein, or multiple proteins, may be used in situations where downstream analysis is desired and/or eliminate proteins that cofound the mass spectrometry analysis.

In some embodiments, targeted depletion may occur by affinity depletion. Affinity depletion refers to methods that deplete a protein of interest from a sample by virtue of its specific binding properties to a molecule. Typically, the molecule is a ligand attached to a solid support, such as a bead, resin, tissue culture plate, etc. (referred to as an immobilized ligand). Immobilization of a ligand to a solid support may also occur after the ligand-protein interaction occurs. Suitable ligands include antibodies, aptamers, and other epitope-binding agents. The molecule may also be a polymer or other material that selectively absorbs a protein of interest. Two or more affinity depletion agents may be combined to sequentially or simultaneously deplete multiple proteins.

Alternatively, protein(s) may be depleted from a sample by a more general method, for example by ultrafiltration or protein precipitation with an acid, an organic solvent or a salt. Generally speaking, these methods are used to reliably reduce high abundance and high molecular weight proteins, which in turn enriches for low molecular weight and/or low abundance proteins and peptides.

In some embodiments, proteins may be depleted from a sample by precipitation. Briefly, precipitation comprises adding a precipitating agent to a sample and thoroughly mixing, incubating the sample with precipitating agent to precipitate proteins, and separating the precipitated proteins by centrifugation or filtration. The resulting supernatant may then be used in downstream applications. The amount of the reagent needed may be experimentally determined by methods known in the art. Suitable precipitating agents include boric acid, perchloric acid, trichloroacetic acid, acetonitrile, methanol, and the like.

As a non-limiting example, proteins may be depleted from a sample by acid precipitation. Following addition of the acid, the sample is mixed well (e.g., by a vortex mixer) and optionally held at a cold temperature, typically for about 10 minutes or longer, to facilitate precipitation. For example, samples may be held for about 10 minutes to about 60 minutes, about 20 minutes to about 60 minutes, or about 30 minutes to about 60 minutes. In other example, samples may be held for about 15 minutes to about 45 minutes, or about 30 minutes to about 45 minutes. In other examples, samples may be held for about 15 minutes to about 30 minutes, or about 20 minutes to about 40 minutes. In other examples, samples are held for about 30 minutes. The sample is then centrifuged at a cold temperature to pellet the precipitated protein, and the supernatant (i.e., the acid soluble fraction), comprising soluble tau, is transferred to a fresh vessel. As used in the above context, a “cold temperature” refers to a temperature of 10° C. or less. For instance, a cold temperature may be about 1° C., about 2° C., about 3° C., about 4° C., about 5° C., about 6° C., about 7° C., about 8° C., about 9° C., or about 10° C. In some embodiments, a narrower temperature range may be preferred, for example, about 3° C. to about 5° C., or even about 4° C. In certain embodiments, a cold temperature may be achieved by placing a sample on ice.

Two or more methods from one or both of the above approaches may be combined to sequentially or simultaneously deplete multiple proteins. For instance, one or more proteins may be selectively depleted (targeted depletion) followed by depletion of high abundance/molecular weight proteins. Alternatively, high abundance/molecular weight proteins may be first depleted followed by targeted depletion of one or more proteins. In still another alternative, high abundance/molecular weight proteins may be first depleted followed by a first round of targeted depletion of one or more proteins and then a second round of targeted depletion of one or more different protein(s) than targeted in the first round. Other iterations will be readily apparent to a skilled artisan.

### (d) Derivatizing Pentasaccharide

Hemiacetals in oligosaccharides are masked aldehydes, and because aldehydes are rare functionalities in cells, reductive amination with 2-aminobenzoic acid (2-AA) is selective for oligosaccharides. The mass spectrometric sensitivity for pentasaccharide (H3N2b) is significantly improved due to introduction of a readily ionizable amino group, and allows detection of previously undetectable plasma and CSF H3N2b. The reductive amination of H3N2b with 2-AA also eliminates α- and β-anomers, and 2-AA-H3N2b appear on chromatograms as a single peak, rather than two α- and β-anomer peaks. More importantly the 2-AA-H3N2b was baseline separated from other isomeric interferences, which is critical to meet the requirement of method selectivity/specificity. In some embodiments, peptide deletion and derivatizing pentasaccharide are performed in a single step. Because minimal sample transfer is used in extraction and derivatization, the pentasaccharide loss during the sample preparation is minimized. The reductive amination condition was carefully optimized to achieve quantitative yield under neutral condition, which minimizes the decomposition of H3N2b. In an exemplary embodiment, the mixture is centrifuged to remove precipitated protein, the excess 2-AA and hydrophobic lipids are removed after supernatant is dried and partitioned between water and methyl tert-butyl ether (MTBE), and the aqueous phase is used directly to LC-MS/MS system.

### (e) Concentrating Pentasaccharide

Another step of the methods disclosed herein comprises concentrating pentasaccharide. A number of methods for concentrating a sample are known in the art and useful according to the present disclosure, including but not limited the methods used in the below examples.

### (f) LC-MS

Another step of the methods disclosed herein comprises performing liquid chromatography-mass spectrometry (LC-MS) with a sample comprising derivatized pentasaccharide to detect and measure the concentration of at least one pentasaccharide. Thus, in practice, the disclosed methods use one or more pentasaccharide to detect and measure the amount of pentasaccharide present in the biological sample.

Suitable LC-MS systems may comprise a <1.0 mm ID column and use a flow rate less than about 100 μl/min. In preferred embodiments, a nanoflow LC-MS system is used (e.g., about 50-100 μm ID column and a flow rate of <1 μL/min, preferably about 100-800 nL/min, more preferably about 200-600 nL/min). In an exemplary embodiment, an LC-MS system may comprise a 0.05 mM ID column and use a flow rate of about 400 nL/min.

Tandem mass spectrometry may be used to improve resolution, as is known in the art, or technology may improve to achieve the resolution of tandem mass spectrometry with a single mass analyzer. Suitable types of mass spectrometers are known in the art. These include, but are not limited to, quadrupole, time-of-flight, ion trap and Orbitrap, as well as hybrid mass spectrometers that combine different types of mass analyzers into one architecture (e.g., Orbitrap Fusion™ Tribrid™ Mass Spectrometer, Orbitrap Fusion™ Lumos™ Mass Spectrometer, Orbitrap Tribrid™ Eclipse™ Mass Spectrometer, Q Exactive Mass Spectrometer, each from ThermoFisher Scientific). In an exemplary embodiment, an LC-MS system may comprise a mass spectrometer selected from Orbitrap Fusion™ Tribrid™ Mass Spectrometer, Orbitrap Fusion™ Lumos™ Mass Spectrometer, Orbitrap Tribrid™ Eclipse™ Mass Spectrometer, or a mass spectrometer with similar or improved ion-focusing and ion-transparency at the quadrupole. Suitable mass spectrometry protocols may be developed by optimizing the number of ions collected prior to analysis (e.g., AGC setting using an orbitrap) and/or injection time. In an exemplary embodiment, a mass spectrometry protocol outlined in the Examples is used.

### III. Diagnosing a Subject

In an aspect, the present disclosure provides a method for measuring GM1-gangliosidosis occurrence or burden in a subject having or suspected of having symptoms of GM1-gangliosidosis, the method comprising (a) measuring pentasaccharide levels, in a urine, a blood sample or a CSF sample obtained from the subject; and (b) using the measurements of (a) to classify the subject as having GM1-gangliosidosis or classifying the subject has having type I, type II, or type III GM1-gangliosidosis. In some embodiments, the methods include measuring H3N2a and/or H3N2b. Suitable methods for measuring pentasaccharides are discussed in section II above and incorporated by reference into this section in their entirety. In some embodiments, the methods of measuring pentasacchride levels includes using a synthetic d₆-H3N2B as an internal standard.

In some of the above embodiments, the measured amount of pentasacchride in the biological sample may be compared to a reference value for the specific pentasacchride measured. The subject levels of pentasacchride in a biological sample are compared to a reference value for pentasacchride, to classify a subject, determine the severity of GM1-gangliosidosis in a subject, determine treatment of a subject, monitor GM1-gangliosidosis in a subject, and/or monitor response to treatment. Generally speaking, a subject may be classified as having an increased or decreased amount of pentasacchride compared to a reference value, wherein an increased amount of pentasacchride is an amount above the reference value and a decreased amount is an amount equal to or below the reference value.

More specifically, the expression level of pentasacchride is compared to the reference value of pentasacchride to determine if pentasacchride in the test sample is different relative to the reference value of the pentasacchride. The term “differential levels” as used herein refers to a difference in the level of the pentasacchride that can be assayed by measuring the level of the pentasacchride.

The term “difference in the level” refers to an increase or decrease in the measurable levels of pentasacchride, for example as measured by the amount of pentasacchride in a biological sample as compared with the measureable level of pentasacchride in a reference sample. In one embodiment, the differential levels can be compared using the ratio of the level of pentasacchride as compared with the level of pentasacchride of a reference sample, wherein the ratio is not equal to 1.0. For example, a pentasacchride is differentially expressed if the ratio of the level of expression of a first sample as compared with a second sample is greater than or less than 1.0. For example, a ratio of greater than 1, 1.2, 1.5, 1.7, 2, 3, 3, 5, 10, 15, 20 or more, or a ratio less than 1, 0.8, 0.6, 0.4, 0.2, 0.1, 0.05, 0.001 or less. In another embodiment, the differential levels is measured using p-value. For instance, when using p-value, a nucleic acid or protein is identified as being differentially expressed between a first sample and a second sample when the p-value is less than 0.1, preferably less than 0.05, more preferably less than 0.01, even more preferably less than 0.005, the most preferably less than 0.001. Depending on the sample used for the reference value, the difference in the level of expression may or may not be statistically significant. For example, if the sample used for reference value is from a subject or subjects diagnosed with GM1-gangliosidosis, then when the difference in the level of is not significantly different, the subject has GM1-gangliosidosis. However, when the difference in the level of expression is significantly different, the subject does not have GM1-gangliosidosis. Alternatively, if the sample used for reference value is from a subject or subjects diagnosed with no disease, then when the difference in the level of expression is not significantly different, the subject does not have GM1-gangliosidosis. However, when the difference in the level of expression is significantly different, the subject has GM1-gangliosidosis.

Any suitable reference value known in the art may be used. For example, a suitable reference value may be the amount of pentasacchride in a biological sample obtained from a subject or group of subjects of the same species that have no signs or symptoms of disease (i.e. GM1-gangliosidosis). In another example, a suitable reference value may be the amount of pentasacchride in a biological sample obtained from a subject or group of subjects of the same species that have not been diagnosed with disease (i.e. GM1-gangliosidosis). In still another example, a suitable reference value may be the amount of pentasacchride in a biological sample obtained from a subject or group of subjects of the same species that have signs or symptoms of GM1-gangliosidosis. In still yet another example, a suitable reference value may be the amount of pentasacchride in a biological sample obtained from a subject or group of subjects of the same species that have been diagnosed with glaucoma. In a different example, a suitable reference value may be the background signal of the assay as determined by methods known in the art. In another different example, a suitable reference value may be the amount of pentasacchride in a non-diseased sample stored on a computer readable medium. In still another different example, a suitable reference value may be the amount of pentasacchride in a diseased sample stored on a computer readable medium. Common forms of computer-readable media include, for example, a floppy disk, a flexible disk, hard disk, magnetic tape, or other magnetic medium, a CD-ROM, CDRW, DVD, or other optical medium, punch cards, paper tape, optical mark sheets, or other physical medium with patterns of holes or other optically recognizable indicia, a RAM, a PROM, and EPROM, a FLASH-EPROM, or other memory chip or cartridge, a carrier wave, or other medium from which a computer can read.

In other examples, a suitable reference value may be the amount of pentasacchride in a reference sample obtained from the same subject. The reference sample may or may not have been obtained from the subject when glaucoma was not suspected. A skilled artisan will appreciate that that is not always possible or desirable to obtain a reference sample from a subject when the subject is otherwise healthy. For example, in an acute setting, a reference sample may be the first sample obtained from the subject at presentation. In another example, when monitoring effectiveness of a therapy, a reference sample may be a sample obtained from a subject before therapy began. In such an example, a subject may have suspected GM1-gangliosidosis but may not have other symptoms of GM1-gangliosidosis or the subject may have suspected GM1-gangliosidosis and one or more other symptom of GM1-gangliosidosis.

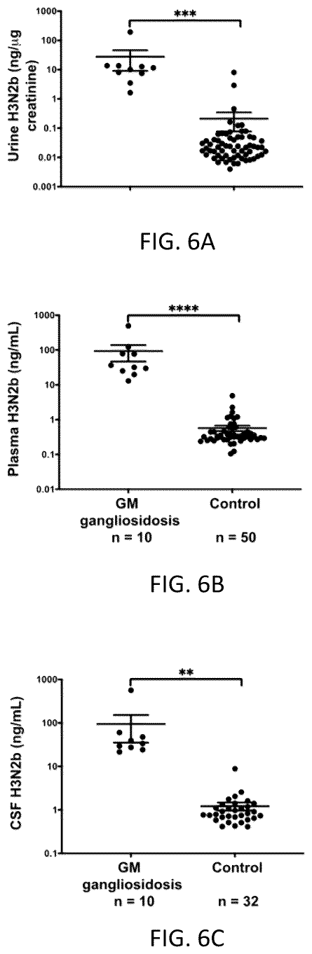

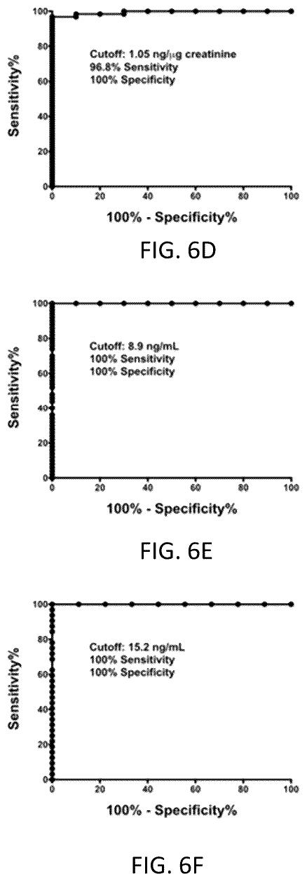

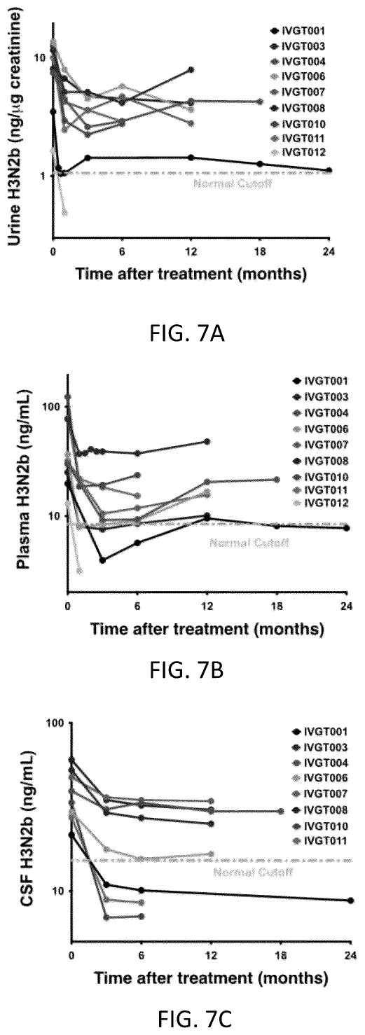

In a specific embodiment, a reference value may be the amount of pentasacchride in a non-diseased sample. For example, a suitable reference value for H3N2b in a urine sample may be about 1 ng/μg creatine or less. In another example, a suitable reference value for H3N2b in a plasma sample may be about 9 ng/mL or less. In still another example, a suitable reference value for H3N2b in a CSF sample may be about 15 ng/mL or less. Specifically, data presented in the Examples shows the normal cutoff values for healthy relative to diseased subjects (see, e.g., FIG. 6-FIG. 7). According to the disclosure, the amount of pentasacchride above the reference value indicates grade of GM1-gangliosidosis. It is to be understood that these values may change due to additional experimental data.

Alternatively or in addition to using a measurement of pentasacchride levels, in any of the above embodiments, a ratio calculated from the measured pentasacchride levels, may be used. Mathematical operations other than a ratio may also be used. For instance, various statistical models (e.g., linear regressions, LME curves, LOESS curves, etc.) in conjunction with other known biomarkers may also be used. Selection of measurements and choice of mathematical operations may be optimized to maximize specificity of the method. For instance, diagnostic accuracy may be evaluated by area under the ROC curve and in some embodiments, an ROC AUC value of 0.7 or greater is set as a threshold (e.g., 0.7, 0.75, 0.8, 0.85, 0.9, 0.95, etc.).

In one example, a method of the present disclosure comprises providing a biological sample obtained from a subject and a) measuring a H3N2b level in the biological sample; (b) classifying the subject based on the measured amount of H3N2b level in step (a) relative to a reference value.

In another example, a method of the present disclosure comprises (a) providing a first and a second biological sample obtained from a subject, wherein “first” and “second” refer to the order in which the samples were collected, and measuring a H3N2b level in the biological sample; (b) calculating the change in the H3N2b level measured; and (c) diagnosing disease progression when the measured H3N2b level increases from the first to the second sample or disease regression when the measured H3N2b level decreases or stays the same from the first to the second sample. The first and the second samples may be collected days, weeks, or months apart.

Methods for measuring H3N2b levels are described in Section II, and incorporated into this section by reference. A skilled artisan will appreciate, however, that the absolute value may vary depending upon the protocol and the source/specifications of internal standards used for absolute quantitation.

In some embodiments of the above, processing a urine sample, a blood sample or a CSF sample from the subject to obtain a first sample may comprise adding an internal standard, protein precipitation, and derivatizing H3N2b.

### IV. Methods of Treatment

The present disclosure also encompasses the use of a pentasacchride measurement described herein to stage a subject's disease progression; to stage a subject's disease pathology; and to select a therapeutic agent, or monitor treatment efficacy of a therapeutic agent for a subject. Accordingly, another aspect of the present disclosure is a method for treating a subject, the method comprising administering to the subject the therapeutic agent.

The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented. In some embodiments, a subject may be diagnosed as having GM1-gangliosidosis. In any of the aforementioned embodiments, a subject may carry one of the gene mutations known to cause GM1-gangliosidosis. In alternative embodiments, a subject may not carry a gene mutation known to cause GM1-gangliosidosis.

In one embodiment, a method for treating a subject as described above may comprise providing a biological sample obtained from a subject and (a) measuring a pentasacchride level in the biological sample; and (b) administering a pharmaceutical composition to the subject when the measured level(s) deviate from a reference value of the same pentasacchride. In another embodiment, a method for treating a subject or monitoring the efficacy of a therapeutic agent, as described above may comprise (a) providing a first biological sample obtained from a subject; (b) administering a pharmaceutical composition to the subject; (c) providing a second biological sample obtained from the subject sometime after administration of the pharmaceutical composition; and (d) measuring in each sample a pentasacchride level, wherein a decrease in the pentasacchride level from the first sample relative to the second sample indicates the pharmaceutical composition is effective or no change or an increase in pentasacchride level indicates the pharmaceutical composition is ineffective.

Treatment strategies contemplated according to the present disclosure include but are not limited to substrate reduction therapy and restoration or replacement of enzymatic activity. Substrate reduction therapy with, for example, miglustat, an inhibitor of the first step in glycosphingolipid biosynthesis, can reduce brain ganglioside, improve neurologic function, and increased lifespan. Miglustat may help slow down or reverse the disease progression in juvenile/adult GM1-gangliosidosis. Restoration or replacement of enzymatic activity may occur through, in non-limiting examples, bone marrow transplantation, pharmacological chaperone, enzyme replacement therapy, and gene therapy. Bone marrow transplantation can normalize leucocyte β-galactosidase levels. A pharmacological chaperone, for example, N-octyl-4-epi-β-valienamine (NOEV), can stabilize the mutant β-galactosidase, and to reduce CNS storage of ganglioside GM1-gangliosidosis with prevention of neurological deterioration in GM1-gangliosidosis. The enhancement of β-galactosidase activity by pharmacological chaperone can be mutation dependent, and the full mutation spectrum for β-galactosidase that can be restored by NOEV is yet to be demonstrated. A major challenge to develop enzyme replacement therapies for GM1-gangliosidosis is the need to deliver corrective enzyme doses to the brain and “hard-to-treat” tissues such as heart, lung and eye. β-Galactosidase:RTB lectin fusion and polymersome vehicles encapsulating β-galactosidase are being explored to address this issue. In preclinical models, adeno-associated viral (AAV) gene therapy is perhaps the most promising in delaying symptom onset, reducing lysosomal storage in the brain and peripheral tissues, and increasing lifespan (1.3-2.3 times in mouse model and >6.7 times in cat model, respectively) with potential for long term efficacy with a single dose. These impressive results have provided the foundation for AAV gene therapy clinical trials. One of major challenges for developing treatments for GM1-gangliosidosis is the difficulty in evaluation of treatment efficacy with clinical endpoints due to the small and heterogeneous patient population as well as slow progression in the non-infantile patients. Therefore, use of validated biomarkers as disclosed herein as outcome measures to predict clinical benefits is critical to evaluating drug efficacy.

Also provided is a process of treating a lysosomal storage disease (e.g., GM1-gangliosidosis) in a subject in need administration of a therapeutically effective amount of a substrate (e.g., pentasaccharide, such as H3N2a or H3N2b) reducing agent or a β-galactosidase modulating agent, so as to reduce the level of substrate or replace or restore β-galactosidase activity.

Methods described herein are generally performed on a subject in need thereof. A subject in need of the therapeutic methods described herein can be a subject having, diagnosed with, suspected of having, or at risk for developing a lysosomal storage disease (e.g., GM1-gangliosidosis). A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of a substrate reducing agent or a β-galactosidase modulating agent is, for example, that amount that would cause the desired therapeutic effect in a subject while minimizing undesired side effects. In various embodiments, an effective amount of a substrate reducing agent or a β-galactosidase modulating agent described herein can substantially inhibit the lysosomal storage disease, slow the progress of the lysosomal storage disease, or limit the development of the lysosomal storage disease.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of a substrate reducing agent or a β-galactosidase modulating agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to reduce the level of substrate or replace or restore β-galactosidase activity.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a substrate reducing agent or a β-galactosidase modulating agent can occur as a single event or over a time course of treatment. For example, a substrate reducing agent or a β-galactosidase modulating agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for the lysosomal storage disease.

A substrate reducing agent or a β-galactosidase modulating agent can be administered simultaneously or sequentially with another agent, such as an antibiotic, an anti-inflammatory, or another agent. For example, a substrate reducing agent or a β-galactosidase modulating agent can be administered simultaneously with another agent, such as an antibiotic or an anti-inflammatory. Simultaneous administration can occur through administration of separate compositions, each containing one or more of a substrate reducing agent or a β-galactosidase modulating agent, an antibiotic, an anti-inflammatory, or another agent. Simultaneous administration can occur through administration of one composition containing two or more of a substrate reducing agent or a β-galactosidase modulating agent, an antibiotic, an anti-inflammatory, or another agent. A substrate reducing agent or a β-galactosidase modulating agent can be administered sequentially with an antibiotic, an anti-inflammatory, or another agent. For example, a substrate reducing agent or a β-galactosidase modulating agent can be administered before or after administration of an antibiotic, an anti-inflammatory, or another agent.

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 □m), nanospheres (e.g., less than 1 □m), microspheres (e.g., 1-100 □m), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Also provided are methods for screening for therapeutics, drugs, or lead compounds for potential use in treating a lysosomal storage disease or for monitoring treatment efficacy or phamacokinetics.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Also provided are kits. Such kits can include an agent or composition described herein and, in certain embodiments, instructions for administration or measuring a pentasacchride. Such kits can facilitate performance of the methods described herein. When supplied as a kit, the different components of the composition can be packaged in separate containers and admixed immediately before use. Components include, but are not limited to individual or combined components useful in the methods disclosed herein. For example, kits may include a stable isotope internal standard (e.g., d₆-H3N2b), reagents for precipitating proteins and/or derivatizing pentasacchrides. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following examples illustrate various iterations of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### Example 1: A Pentasaccharide Biomarker for Monitoring Pharmacodynamic Response to Gene Therapy in GM1 Gangliosidosis

GM1 gangliosidosis is a rare, fatal, neurodegenerative disease caused by mutations in the GLB1 gene, in which β-galactosidase enzyme activity is deficient and glycoconjugates with a terminal β-galactose accumulate. Delay of symptom onset, reduction of storage in the brain and peripheral tissues, and increase of lifespan were achieved in a GM1 gangliosidosis cat model after adeno-associated viral (AAV) gene therapy treatment, providing the basis for early-stage AAV gene therapy trials. As the disorder is slowly progressive, particularly in the late-infantile and juvenile forms, the availability of validated biomarkers would greatly improve assessment of therapeutic efficacy of the AAV gene therapy. The present example identifies two pentasaccharide biomarkers, H3N2a and H3N2b, that were elevated more than 20-fold in patient plasma, cerebrospinal fluid (CSF), and urine. Only H3N2b was detectable in the central nervous system of cat model, and it was reduced after AAV gene therapy treatment. Through mass spectrometric analyses, chemical and enzymatic degradations, the structures of the pentasaccharide biomarkers were elucidated. The structure of H3N2b was confirmed by comparison of liquid chromatography-tandem mass spectrometry of endogenous and synthetic compounds. Synthesis of a stable isotope-labeled internal standard enabled development of fully validated assays and accurate quantification of this biomarker in biospecimens. Reduction of H3N2b in urine, plasma, and CSF samples from the participants of a Phase 1/2 intravenous AAV9 gene therapy trial was demonstrated, suggesting that this pentasaccharide is a useful pharmacodynamic biomarker for monitoring therapeutic response and has implications for accelerating drug approval in this rare genetic disease.

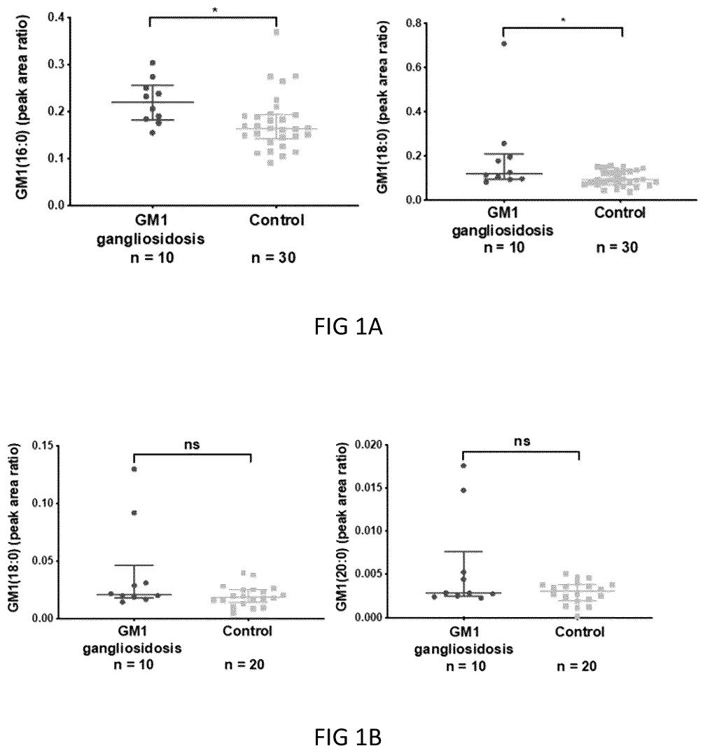

Ganglioside GM1 in human biofluids: A wide range of potential biomarkers have been explored in the GM1 gangliosidosis cat model to assess clinical efficacy of AAV gene therapy. CSF ganglioside GM1 appeared the most promising exhibiting >30-fold elevation in untreated GM1 gangliosidosis cats, and reduction in response to AAV gene therapy treatment. Ganglioside GM1 in plasma and CSF was compared from type II GM1 gangliosidosis and control human subjects. The elevation in plasma ganglioside GM1 was less than 2-fold, with only the most abundant ganglioside GM1 species in the plasma, GM1(16:0) and GM1(18:0), showing significant elevation in GM1 gangliosidosis patients (FIG. 1A). These results were in agreement with only moderate elevation of ganglioside GM1 in dried blood spots from GM1 gangliosidosis patients. By contrast, there were no significant differences in any of the ganglioside GM1 species in CSF between GM1 gangliosidosis patients and controls (FIG. 1B).

Discovery of oligosaccharides as GM1 gangliosidosis biomarkers: Several oligosaccharides have been found to be significantly elevated in urine from GM1 gangliosidosis patients and used for diagnosis of this disorder (11-15). Oligosaccharides in urine, plasma and CSF samples were profiled. A simple and sensitive liquid chromatography-tandem mass spectrometry (LC-MS/MS) method for profiling oligosaccharides was developed, in which reductive amination of oligosaccharides with 2-aminobenzoic acid (2-AA) and sodium cyanoborohydride (NaBH₃CN) was used to increase its retention on reversed-phase high performance liquid chromatography (HPLC) column and mass spectrometric sensitivity.

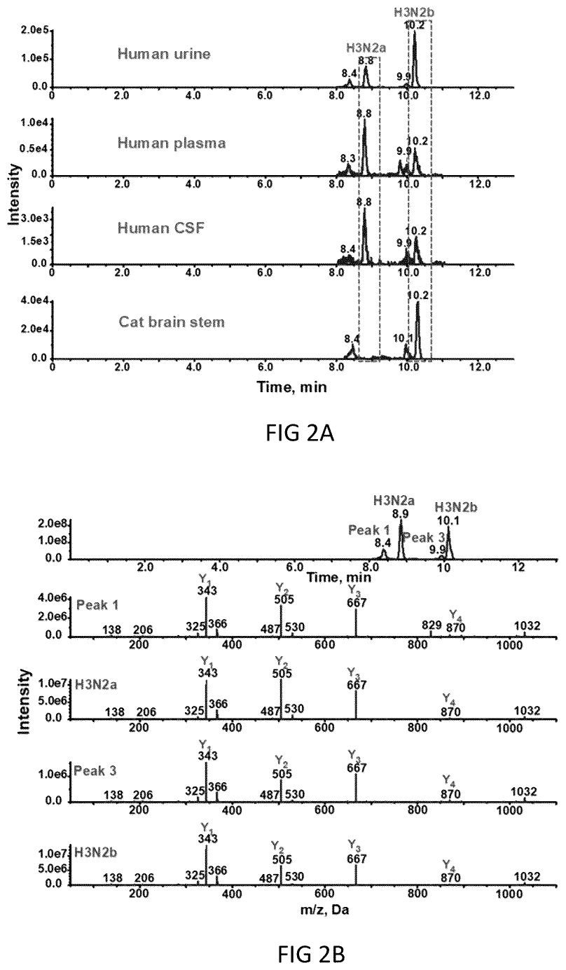

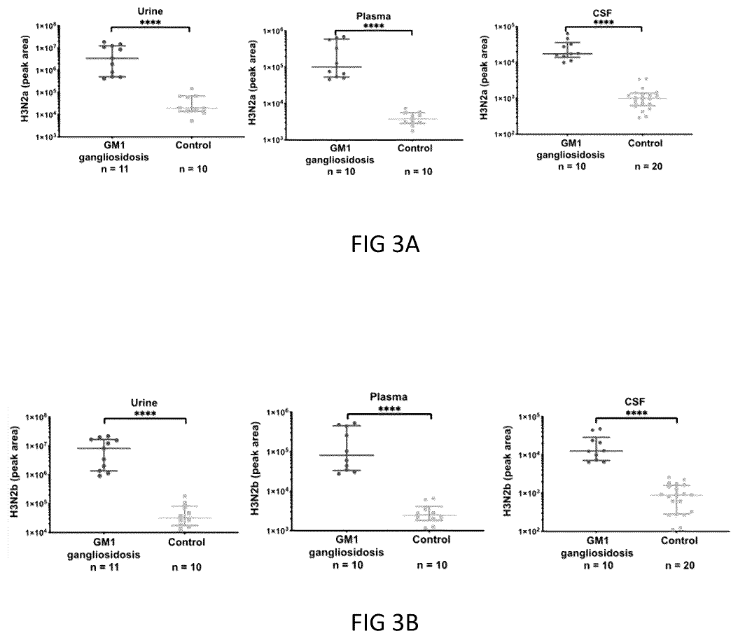

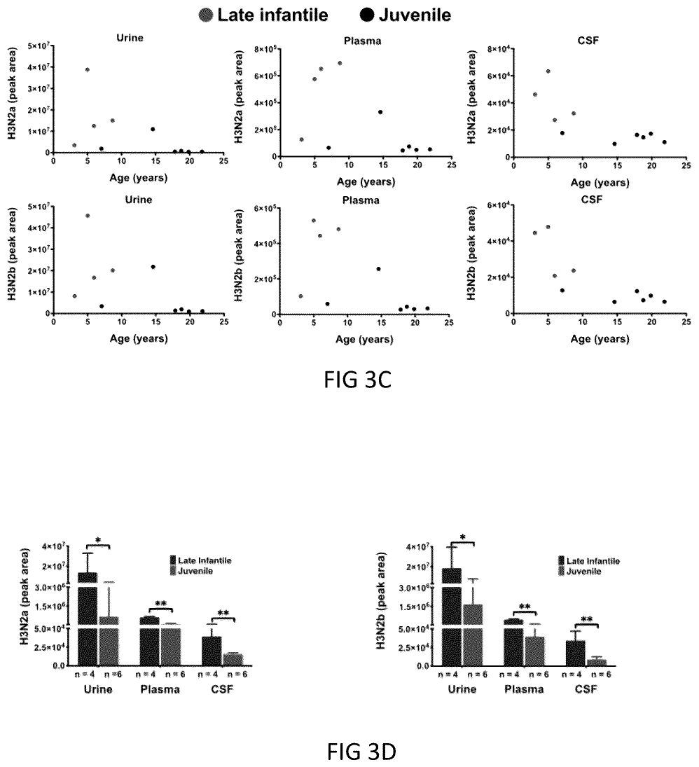

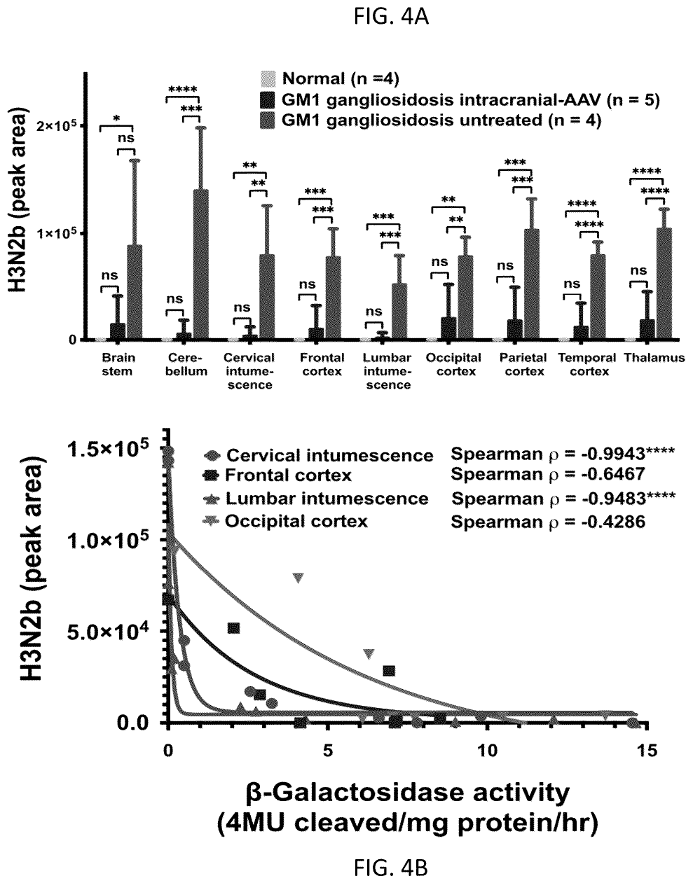

A pair of pentasaccharides (H3N2a and H3N2b) were identified that were significantly elevated in GM1 gangliosidosis urine samples, where H is hexose, and N is N-acetylhexosamine. The pentasaccharides H3N2a and H3N2b were detectable in patient plasma and CSF (FIG. 2A). There are unknown interfering isomers (peak 1 and 3 in FIG. 2B) of H3N2a and H3N2b in human samples, which have not been reported previously. H3N2a and H3N2b were elevated >20-fold in urine, plasma, and CSF samples from type II (late infantile and juvenile) GM1 gangliosidosis patients (FIG. 3A and FIG. 3B). While there was no significant correlation between age and H3N2a and H3N2b levels, late infantile patients showed higher H3N2a and H3N2b levels compared to juvenile patients (FIG. 3C and FIG. 3D). Lower residual β-galactosidase enzyme activity was reported in late infantile compared to juvenile patients, suggesting that the H3N2a and H3N2b levels are negatively correlated with residual β-galactosidase enzyme activity. There was no significant difference in H3N2a and H3N2b levels between male and female patients. It was further found that the H3N2b but not H3N2a was detectable and elevated in the CNS of the GM1 gangliosidosis cat model, including brain stem, cerebellum, spinal cord (cervical intumescence, lumbar intumescence), frontal cortex, occipital cortex, parietal cortex, temporal cortex, and thalamus regions (brain stem as an example shown in FIG. 2A). H3N2b was elevated >100-fold in all of these regions of the cat CNS (FIG. 4A). An inverse correlation between H3N2b levels and β-galactosidase enzyme activities was observed in cat cervical intumescence, lumbar intumescence, frontal cortex, and occipital cortex (FIG. 4B). H3N2b was used to assess AAVrh8 gene therapy treatment efficacy in cat CNS. The pentasaccharide biomarker was significantly reduced after AAVrh8 gene therapy treatment in all regions except for brain stem, which was more difficult to treat by intracranial injection of AAVrh8 (FIG. 4A). The finding that H3N2b is a common biomarker for the cat model and for GM1 gangliosidosis patients suggests that the pentasaccharide may be able to assist in the translation of new treatments from the cat model into patients.

Structural identification of H3N2a and H3N2b: The H3N2a and H3N2b oligosaccharides were detected previously in urine from patients with GM1 gangliosidosis disease and Morquio B syndrome and characterized as underivatized- and permethylated-oligosaccharides on different MS platforms. Only the structure of H3N2a isolated from the urine of a Morquio B patient had been confirmed with proton nuclear magnetic resonance spectroscopy (¹HNMR). To obtain structural information for the biomarkers, 2-AA-H3N2a and 2-AA-H3N2b were isolated from HPLC fractions. It was found that structural information obtained from underivatized- and permethylated-forms was different from 2-AA derivatives. Rigorous structure characterization was performed to identify the structures of H3N2a and H3N2b, including tandem mass spectrometry, chemical and enzymatic degradations. The product ion spectra (FIG. 2B) of [M+H]⁺, [M−H]⁻, and [M+Na]⁺ ions of 2-AA derivatives indicate that the sequences of both oligosaccharides are ⁵Hexose-⁴N-acetylhexosamine-³Hexose-²Hexose-¹N-acetylhexosamine. Sodium periodate (NaIO₄) oxidation products of 2-AA-H3N2a and 2-AA-H3N2b suggested that the linkages of H3N2a and H3N2b were ⁵Hexose-(1→4)-⁴N-acetylhexosamine-(1→2)-³Hexose-(1→6)-²Hexose-(1→4)-1N-acetylhexosamine and ⁵Hexose-(1→4)-⁴N-acetylhexosamine-(1→2)-³Hexose-(1→3)-²Hexose-(1→4)-1N-acetylhexosamine, respectively. The stepwise digestion of 2-AA-H3N2a and 2-AA-H3N2b with β1,4-galactosidase, β-N-acetylglucosaminidase, α-1-2,3,6-mannosidase, and β-mannosidase confirmed the H3N2a and H3N2b were ⁵galactose-(β1→4)-⁴N-acetylglucosamine-(β1→2)-³mannose-(α1→6)-²mannose-(β1→4)-1N-acetylhexosamine and ⁵galactose-(β1→4)-⁴N-acetylglucosamine-(β1→2)-³mannose-(α1→3)-²mannose-(β1→4)-¹N-acetylhexosamine, respectively. The reducing terminus of both 2-AA-H3N2a and 2-AA-H3N2b was identified as 2-AA-N-acetylglucosamine that was confirmed with a standard compound. The H3N2a and H3N2b were identified as O-β-D-galactopyranosyl-(1→4)-O-(2-acetamido-2-deoxy-β-D-glucopyranosyl)-(1→2)-O-α-D-mannopyranosyl-(1→6)-O-β-D-mannopyranosyl-(1→4)-2-acetamido-2-deoxy-α,β-D-glucopyranose and O-β-D-galactopyranosyl-(1→4)-O-(2-acetamido-2-deoxy-β-D-glucopyranosyl)-(1→2)-O-α-D-mannopyranosyl-(1→3)-O-β-D-mannopyranosyl-(1→4)-2-acetamido-2-deoxy-α,β-D-glucopyranose, respectively. They are moieties of complex N-glycan.

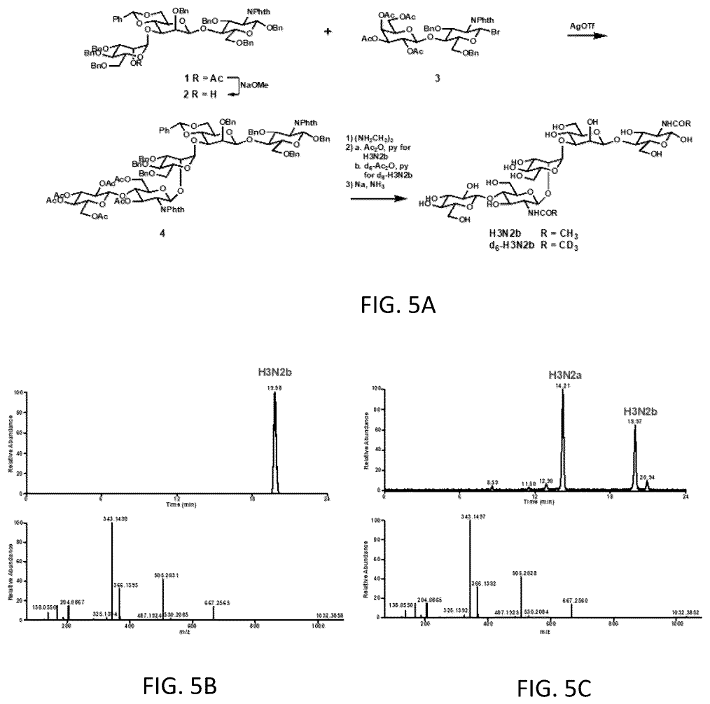

Synthesis of H3N2b and d6-H3N2b: H3N2b was chosen for further biomarker development, which is the common biomarker observed in the GM1 cat model and patients. As no commercial reference standard of H3N2b and its stable isotope-labeled analog as internal standard were available, H3N2b and d6-H3N2b were synthesized from acceptor 1 and donor 3 in 23% and 16.5% overall yields over 5 steps (FIG. 5A). H3N2b and d6-H3N2b with chromatographic purity >99% (H3N2b is shown in FIG. 5B) were obtained after purification with HPLC, which were determined after derivatization with 2-AA. The retention time and tandem mass spectrum of synthetic H3N2b were the same as endogenous H3N2b (FIG. 5C) after derivatization with 2-AA, confirming the proposed structure of H3N2b.

Validation of LC-MS/MS assays for determination of H3N2b in human urine, plasma, and CSF: After synthesis of H3N2b and internal standard d6-H3N2b, the LC-MS/MS conditions were further optimized and the methods for quantification of H3N2b in human urine, plasma, and CSF validated according to FDA Bioanalytical Method Validation Guidance. As H3N2b is an endogenous analyte, water was used as a surrogate matrix for standard curves for urine, plasma, and CSF. The methods showed good linearity and parallelism, selectivity and specificity, sensitivity, accuracy, precision, and reproducibility, stability, carryover, and dilution integrity. The validated methods allowed us to reliably quantify H3N2b in clinical biospecimens.

Establishment of normal cutoffs for human urine, plasma, and CSF H3N2b: Urine samples from 10 untreated GM1 gangliosidosis patients and 63 control subjects, plasma samples from 10 untreated GM1 gangliosidosis patients and 50 control subjects, and CSF samples from 10 untreated GM1 gangliosidosis patients and 32 control subjects were analyzed. The reference ranges for GM1 gangliosidosis urine, control urine, GM1 gangliosidosis plasma, control plasma, GM1 gangliosidosis CSF, and control CSF, were 1.64-193 ng/μg creatinine, 0.004-8.04 ng/μg creatinine, 13-496 ng/mL, <1-4.88 ng/mL, 21.6-563 ng/mL, and <1-8.84 ng/mL, respectively (FIG. 6A-FIG. 6C). The cut-offs of 1.05 ng/μg creatinine, 8.9 ng/mL, and 15.2 ng/mL for urine, plasma, and CSF were established from receiver operator characteristic (ROC) curves. Sensitivities of 96.8%, 100%, and 100% were obtained to distinguish GM1 gangliosidosis from control based on urine, plasma, and CSF H3N2b, respectively. Specificities of 100% were observed using urine, plasma, and CSF H3N2b cutoffs (FIG. 6D-FIG. 6F).

Pharmacodynamic response of H3N2b in GM1 gangliosidosis patients receiving AA V gene therapy: To evaluate the response of H3N2b to intravenous AAV9-mediated GLB1 gene therapy in GM1 gangliosidosis patients, H3N2b levels were analyzed in pre- and post-treatment urine, plasma, and CSF samples collected from 8 participants with late infantile and juvenile onset GM1 enrolled in the Phase 1/2 gene therapy trial (NCT03952637) and one juvenile onset child who received the treatment under an expanded access protocol. Significant decreases in urine, plasma, and CSF H3N2b were detectable by 1, 1, and 3 months after treatment, respectively (FIG. 7). Urine, plasma, and CSF H3N2b levels were reduced 32-80%, 28-81%, and 28-79% at the last time point, respectively. H3N2b levels decreased to the normal range (derived in FIG. 6) in urine of participants IVGT001 and IVGT012, plasma of participants IVGT001 and IVGT012, and CSF of participants IVGT001, IVGT010 and IVGT011. These data indicate that gene therapy to reduce lysosomal storage in both the CNS and somatic tissues was accompanied by significant changes in the H3N2b biomarker. In participants IVGT010 and IVGT011 who received the high dose of gene therapy, the CSF H3N2b levels were reduced to lower levels compared to those receiving the low dose, suggesting that high dose led to higher efficacy in CNS. Among the 9 participants with decreases in the H3N2b biomarker, 7 of 9 had stabilization or improvement on the Vineland Adaptive Behavior Scale Version V. 3; 8 of 9 were stable on floor and upright mobility assessments; 3 of 4 had stability or improvement in total brain volume by magnetic resonance imaging (MRI); and 7 of 9 showed consistent small improvement on the Clinical Global Impressions Scale.

DISCUSSION: Drug development in rare neurodegenerative diseases is challenging due to the small and heterogeneous patient population as well as slow progression in the non-infantile patients. Use of non-invasive biomarkers is critical to accelerate treatment development. Numerous potential biomarkers for GM1 gangliosidosis have previously been reported and several have been explored in the GM1 gangliosidosis cat model to assess response to therapy. Of these, CSF ganglioside GM1 appeared the most promising, exhibiting more than 30-fold elevation in untreated GM1 gangliosidosis cats and reduction following AAV gene therapy treatment. In the present study, however, it was found that the application of ganglioside GM1 in clinical studies was limited by insufficient dynamic range between GM1 gangliosidosis and control, as there was less than 2-fold elevation in the CSF and plasma of GM1 gangliosidosis patients; there was overlap between GM1 gangliosidosis and controls, and GM1 in urine was undetectable. Given the advances in gene therapy for GM1 gangliosidosis, identification of additional clinically relevant biomarkers are urgently needed to assess treatment response.

Reports have shown that AAV gene therapy in the GM1 gangliosidosis cat model restored β-galactosidase activity, leading to extraordinary extension in lifespan and improvement of neurological symptoms. Based on these preclinical results, the Phase 1/2 clinical trial of intravenous AAV9 gene therapy (NCT03952637) for type I and II GM1 gangliosidosis was initiated in May 2019, and intracisternal AAVrh10 (NCT04273269) and AAVHu68 (NCT04713475) gene therapies for type I and II GM1 gangliosidosis are underway. To facilitate the translation of AAV gene therapy developed in animal models to patients, biomarker discovery efforts were undertook in this disorder. Since oligosaccharides had been shown previously to be significantly elevated in urine from GM1 gangliosidosis patients, oligosaccharides in biospecimens from a natural history study at NIH (NCT03952637) and the GM1 gangliosidosis cat model were profiled. Two pentasaccharides, H3N2a and H3N2b, re found elevated >20-fold in human plasma, CSF, and urine. For reasons yet to be determined, however, only H3N2b was detectable in the CNS of the GM1 gangliosidosis cats. H3N2b is a potential translatable biomarker for assessing AAV treatment efficacy in the cat model and patients. These pentasaccharides had previously been proposed as diagnostic biomarkers. However, due to lack of authentic standards, ill-defined structures and detection of H3N2a and H3N2b as a mixture, the oligosaccharide assays were semi-quantitative and no reference ranges were established, which limited their utility as pharmacodynamic biomarkers. Here, preliminary assignments of the pentasaccharide structures were achieved through mass spectrometric analyses, chemical and enzymatic degradations, followed by synthesis of standard compound of H3N2b, and the proposed H3N2b structure was confirmed by comparison of endogenous and synthetic compounds using LC-MS/MS. With synthetic H3N2b at hand, fully validated assays were developed to accurately quantify H3N2b in the samples collected from clinical studies and to establish the reference ranges for normal and GM1 gangliosidosis. This work paves the way for application of H3N2b as a pharmacodynamic biomarker for AAV gene therapy or any treatment that increases β-galactosidase activity.

The present example provides strong support for the H3N2b pentasaccharide as a pharmacodynamic biomarker for response to therapy in GM1 gangliosidosis patients. This natural substrate of β-galactosidase is elevated in GM1 gangliosidosis, and its level is inversely proportional to enzyme activity. It was found that H3N2b levels were sharply reduced in the CNS of GM1 gangliosidosis cats in response to intracranial AAV gene therapy. Furthermore, the biomarker was also reduced in urine, plasma and CSF samples from the patients receiving intravenous AAV9-GLB1 gene therapy. The dose-dependent reduction of CSF H3N2b is consistent with an ability of the AAV9 vector to cross blood brain barrier and transduce cells in the CNS following intravenous administration, strongly supporting that systemic AAV9 administration will be able to effectively restore functional β-galactosidase for the treatment of both the neurological and somatic disorders of GM1 gangliosidosis. This contrasts with other GM1 gangliosidosis biomarkers that can only serve as either peripheral or CNS biomarkers.

H3N2b offers an expanded dynamic range to evaluate treatment efficacy compared to GM1 gangliosides, which were elevated less than two-fold in plasma and were not elevated at all in CSF of GM1 gangliosidosis patients compared to controls. In fact, it is important to note that patient CSF has no elevation of GM1 ganglioside but ˜20-fold increases in H3N2b, which will require future investigations of H3N2b's role in pathogenesis. Notably, H3N2b is readily quantified in biofluids such as urine, plasma, and CSF, making it an ideal non-invasive biomarker for serially monitoring response to treatment. These features will facilitate use of H3N2b as a pharmacodynamic biomarker in gene therapy trials for GM1 gangliosidosis. It is also possible that H3N2b could serve as pharmacodynamics biomarker beyond gene therapy for treatments targeted at augmenting β-galactosidase activity, such as marrow transplantation (34), pharmacological chaperones, and enzyme replacement therapy.

MATERIALS AND METHODS: The goal of the example was to identify biomarkers for monitoring pharmacodynamic response to gene therapy in GM1 gangliosidosis. The plasma, CSF, and urine samples from GM1 gangliosidosis patients in a natural history study (NCT03952637) and the Phase 1/2 study of intravenous gene transfer vector AAV9/GLB1 for GM1 gangliosidosis (NCT03952637), and age and gender-matched control subjects were obtained. The CNS samples were collected from normal, GM1 gangliosidosis, and AAV-treated GM1 gangliosidosis cats. Ganglioside GM1 and oligosaccharides were profiled in human biofluid samples from the natural history study, and two pentasaccharides H3N2a and H3N2b were selected as candidate biomarkers. The evaluation of response of both biomarkers to AAV gene therapy in the cat model led to H3N2b as a potential pharmacodynamic biomarker. The response of H3N2b in patients to AAV gene therapy was confirmed after its structure was identified, standard compound and internal standard were synthesized, and fully validated assays were developed.

Chemicals and reagents: The formic acid, methanol, acetonitrile, diethylamine, hexafluoro-2-propanol, 2-AA, NaBH₃CN, MTBE, NaIO₄, NaBH₄, ammonium acetate, ammonium hydroxide solution, ethylene glycol, acetic acid, and all the reagents and solvents used in the synthesis of H3N2b and d₆-H3N2b were purchased from Sigma-Aldrich (St. Louis, MO). The β(1-4)-galactosidase (Streptococcus pneumoniae), β-N-acetylhexosaminidase (recombinant from Streptococcus pneumoniae, expressed in E. coli), α(1-2,3,6)-mannosidase (Jack Bean), β-mannosidase (Helix pomatia), and the buffers were purchased from ProZyme (Hayward, CA). The d₃-GM1(18:0) was provided by Matreya (State College, PA). Milli-Q ultrapure water was prepared in-house with a Milli-Q Integral Water Purification System (Billerica, MA).

Human subjects: Human studies adhered to the principles of the Declaration of Helsinki, as well as to Title 45, US Code of Federal Regulations, Part 46, Protection of Human subjects. De-identified urine, plasma, and CSF samples were collected from GM1 gangliosidosis natural history study (NCT03952637) at NHGRI and from the Phase 1/2 study of intravenous gene transfer vector AAV9/GLB1 (NCT03952637) at NHGRI. Control urine, plasma, and CSF samples were obtained from anonymized residual samples at St. Louis Children's Hospital. The GM1 gangliosidosis patients were diagnosed with genetic testing and β-gatactosidase activity. The control subjects were confirmed based on the medical record. The demographic characteristics of human subjects are given in Table 1. Collection and analysis of de-identified human samples were approved by the institutional review boards at Washington University and NHGRI.

TABLE 1
Demographic characteristics of patients

| | Sample | | GM1 | |
|---|---|---|---|---|
| Cohort | type | Patient type | gangliosidosis | Control |
| Evalution of | Plasma | Age (range, median) | 3-20, 11 | 1-18, 10 |
| ganglioside | | Male (n) | 5 | 20 |
| GM1 | | Female (n) | 5 | 10 |
| | | Late infantile (n) | 4 | |
| | | Juvenile (n) | 6 | |
| | CSF | Age (range, median) | 3-20, 11 | <1-20, 6 |
| | | Male (n) | 5 | 9 |
| | | Female (n) | 5 | 11 |
| | | Late infantile (n) | 4 | |
| | | Juvenile (n) | 6 | |
| Discovery of | Urine | Age (range, median) | 3-20, 11 | 1-17, 13 |
| oligosaccharide | | Male (n) | 5 | 5 |
| biomarkers | | Female (n) | 5 | 5 |
| | | Late infantile (n) | 4 | |
| | | Juvenile (n) | 6 | |
| | Plasma | Age (range, median) | 3-20, 11 | 1-18, 11 |
| | | Male (n) | 5 | 5 |
| | | Female (n) | 5 | 5 |
| | | Late infantile (n) | 4 | |
| | | Juvenile (n) | 6 | |
| | CSF | Age (range, median) | 3-20, 11 | <1-20, 6 |
| | | Male (n) | 5 | 9 |
| | | Female (n) | 5 | 11 |
| | | Late infantile (n) | 4 | |
| | | Juvenile (n) | 6 | |
| Establishment | Urine | Age (range, median) | <1-10, 6 | <1-26, 10 |
| of H3N2b | | Male (n) | 4 | 19 |
| reference range | | Female (n) | 6 | 44 |
| and Phase 1/2 | | Late infantile (n) | 4 | |
| trial | | Juvenile (n) | 6 | |
| | | Low dose: 1.5 × 103 | 7 | |
| | | vg/kg (n) | | |
| | | High dose: 4.5 × 103 | 3 | |
| | | vg/kg (n) | | |
| Establishment | Plasma | Age (range, median) | <1-10, 6 | <1-23, 9 |
| of H3N2b | | Male (n) | 4 | |
| reference range | | Female (n) | 6 | |
| and Phase 1/2 | | Late infantile (n) | 4 | 21 |
| trial | | Juvenile (n) | 6 | 29 |
| | | Low dose: 1.5 × | 7 | |
| | | 10(13) vg/kg (n) | | |
| | | High dose: 4.5 × | 3 | |
| | | 10(13) vg/kg (n) | | |
| Establishment | CSF | Age (range, median) | <1-10, 6 | <1-19, 8 |
| of H3N2b | | Male (n) | 4 | 18 |
| reference range | | Female (n) | 6 | 14 |
| and Phase 1/2 | | Late infantile (n) | 4 | |
| trial | | Juvenile (n) | 6 | |
| | | Low dose: 1.5 × | 7 | |
| | | 10(13) vg/kg (n) | | |
| | | High dose: 4.5 × | 3 | |
| | | 10(13) vg/kg (n) | | |

Experimental animals: GM1 gangliosidosis cats were raised at Auburn University College of Veterinary Medicine. All animal procedures were approved by the Institutional Animal Care and Use Committee at Auburn University. Treatment of GM1 gangliosidosis cat with intracranial AAVrh8 gene therapy and collection of brain stem, cerebellum, spinal cord (cervical intumescence, lumbar intumescence), frontal cortex, occipital cortex, parietal cortex, temporal cortex, and thalamus were described previously.

Analysis of H3N2b in urine, plasma, and CSF samples from Phase 1/2 intravenous AAV gene therapy trial: Samples consisting of calibration standards in duplicate, a blank, a blank with internal standard, quality control (QC) samples at the levels of low (LQC), middle (MQC), and high (HQC), and unknown clinical samples were analyzed. The total number of QC samples was at least 5% of that of unknown clinical samples. The standard curve covered the expected unknown sample concentration range, and samples that exceeded the highest standard could be diluted and re-assayed. In the dilution sample re-assay, a dilution QC (DQC) in triplicate was also included in the analytical run. The LC-MS/MS run acceptance criteria of FDA guidance were met. The urine H3N2b was normalized to creatinine that was measured with procedure reported previously.

Profiling of ganglioside GM1 in human plasma and CSF samples from GM1 gangliosidosis natural history study: Human plasma, CSF, and urine samples (50 μL) were aliquoted into 2 mL polypropylene tubes (VWR, West Chester, PA). To each tube d3-GM1(18:0) (400 ng/mL) in methanol (200 μL) was added. The mixtures were vortexed for 3 min and centrifuged for 10 min at 9400 g. The supernatants were transferred to 1.2 mL glass inserts (VWR, West Chester, PA) for LC-MS/MS assay on a Shimadzu Prominence HPLC system (Columbia, MD) coupled with a 4000QTRAP mass spectrometer (AB Sciex, Framingham, MA). The GM1 were separated on an ACE Super C18 column (4.6×50 mm, 3 μm) (Mac-Mod Analytical, Chadds Ford) protected with a SecurityGuard Gemini C18 guard column (4×3 mm) (Phenomenex, Torrance, CA). The mobile phases consisted of in 2.9 mM diethylamine and 20 mM hexafluoro-2-propanol in water (solvent A) and methanol-tetrahydrofuran (97:3) (solvent B), and the flow rate was 1 mL/min. The gradient was as follows: 0 to 4 min, 85 to 100% solvent B; 4 to 6 min, 100% solvent B; 6 to 6.1 min, 100 to 85% solvent B; 6.1 to 8 min, 60% solvent B. The eluate was directed into the mass spectrometer for data acquisition from 1.8 to 4 min; elsewhere, eluate was sent to waste to minimize source contamination. The injection volume was 4 μL, and the total run-time was 8 min. The electrospray ionization (ESI) source temperature was 500° C.; the electrospray voltage was −4500 V. The multiple reaction monitoring (MRM) transitions of m/z 1516.8 to 290.1, 1544.9 to 290.1, 1572.9 to 290.1, 1547.9 to 290.1 were used to detect GM1(16:0), GM1(18:0), GM1(20:0), d3-GM1(18:0), respectively. The dwell time, declustering potential (DP), collision energy (CE), entrance potential (EP), and the collision cell exit potential (CXP) were 50 ms, −210 eV, −97 eV, −10 eV, and −10 eV, respectively, for all the MRM transitions. Data were acquired and analyzed by Analyst software (version 1.6.3).

Profiling of oligosaccharides in human plasma, CSF, urine and cat CNS samples from GM1 gangliosidosis natural history study: The cat CNS tissues (100-300 mg) were homogenized in water (4 mL/g wet tissue) in 2 mL Omni homogenization tubes containing 8 mm ceramic beads and processed on the Bead Ruptor 24 (Omni International, Kennesaw, GA) for two 30 second cycles at 5.65 m/s with a 45 second dwell. Human plasma, CSF, urine, cat tissue homogenate samples (50 μL) were aliquoted into 2 mL polypropylene tubes (VWR, West Chester, PA). To each tube 2-AA (30 mg/mL) and NaBH₃CN (20 mg/mL) in methanol (200 μL) was added. The mixtures were vortexed for 3 min, heated at 80° C. for 1 hour, and centrifuged for 10 min at 9400 g. The supernatants were transferred to 1.2 mL glass inserts (VWR, West Chester, PA) and dried with nitrogen flow at 50° C. The residues were partitioned between water (200 μL) and MTBE (600 μL), and aqueous phases were transfer to new 1.2 mL glass inserts for LC-MS/MS assay on a Shimadzu Prominence HPLC system (Columbia, MD) coupled with a 6500QTRAP+ mass spectrometer (AB Sciex, Framingham, MA). The oligosaccharides were separated on an ACE C18 column (4.6×150 mm, 3 μm) (Mac-Mod Analytical, Chadds Ford) protected with a SecurityGuard C18 guard column (4×3 mm) (Phenomenex, Torrance, CA). The mobile phases consisted of 0.1% formic acid in water (solvent A) and 0.1% formic acid in methanol-acetonitrile (4:1) (solvent B), and the flow rate was 1 mL/min. The gradient was as follows: 0 to 10 min, 3 to 40% solvent B; 10 to 10.1 min, 40 to 100% solvent B; 10.1 to 11 min, 100% solvent B; 11 to 11.1 min, 100 to 3% solvent B; 11.1 to 13 min, 3% solvent B. The eluate was directed into the mass spectrometer for data acquisition from 8 to 11 min; elsewhere, eluate was sent to waste to minimize source contamination. The injection volume was 2 μL, and the total run-time was 13 min. The ESI source temperature was 500° C.; the electrospray voltage was 5500 V; the DP was 75 V; the EP and the CXP were 10 and 10 V, respectively. The collision and curtain gas were set at medium and 30, respectively. Both desolvation gas and nebulizing gas were set at 35 L/min. The CEs for MRM transitions of m/z 1032.4 to 667.3 (quantifier for 2-AA derivatized H3N2a and H3N2b) and m/z 1032.4 to 343.1 (qualifier for 2-AA derivatized H3N2a and H3N2b) were 30 and 45 eV, respectively. The dwell time was set at 50 ms for both MRM transitions. Data were acquired and analyzed by Analyst software (version 1.6.3).

Assays of β-galactosidase activity: The frozen sections (50 μm) were cut from cat cervical intumescence, frontal cortex, lumbar intumescence, and occipital cortex issue and homogenized manually in 50 mM citrate phosphate buffer, pH 4.4 (50 mM citric acid, 50 mM disodium hydrogen phosphate, 10 mM sodium chloride) containing 0.1% TritonX and 0.05% bovine serum albumin, followed by 2 freeze-thaw cycles and centrifugation at 15,700 g for 5 minutes at 4° C. The activity of β-galactosidase was measured using synthetic fluorogenic substrate 4MU-β-D-galactoside as previously described. Specific activity normalized to protein concentration by the Lowry method was expressed as nmol 4MU/mg protein/hr.

Liquid chromatography-enhanced product ion scan (LC-EPI) of 2-AA derivatized H3N2a and H3N2b: The HPLC condition was same as that used to profile oligosaccharides in human plasma, CSF, urine and cat CNS samples. The ESI source temperature was 500° C.; the electrospray voltages for both 2-AA derivatized H3N2a and H3N2b ions at m/z 1032.4 ([M+H]⁺), 1054.4 ([M+Na]⁺), 1030.4 ([M−H]⁻) were 5500, 5500, −4500V, respectively; the DP were 80, 80, −80 V for [M+H]⁺, [M+Na]⁺, [M−H]⁻ ions of both 2-AA derivatized H3N2a and H3N2b, respectively; the mass scan range was m/z 50-1400; scan speed was 10000 u/s; and dynamic fill time was used; the CE for EPI scan of [M+H]⁺, [M+Na]⁺, [M−H]⁻ ions of both 2-AA derivatized H3N2a and H3N2b were 40, 80, −90 eV, respectively. The collision and curtain gas were set at high and 30, respectively. Both desolvation gas and nebulizing gas were set at 35 L/min. Data were acquired and analyzed by Analyst software (version 1.6.3).

Enzymatic degradation of 2-AA derivatized H3N2a and H3N2b: The HPLC fractions of 2-AA derivatized H3N2a and H3N2b were dissolved in 50 μL of β(1-4)-galactosidase (0.2 U/mL) in ammonium acetate buffer (0.05 M, pH 6.0) and incubated at 37° C. for 3 hours. The 2 μL of mixture was diluted with 18 μL of water followed by LC-MS/MS assay. The rest mixture was dried with nitrogen flow at 50° C., and the residue was dissolved in 50 μL of β-N-acetylhexosaminase (4 U/mL) in ammonium acetate buffer (0.05 M, pH 6.0) followed by incubation at 37° C. for 16 hours. The 2 μL of mixture was diluted with 18 μL of water followed by LC-MS/MS assay. The rest mixture was dried with nitrogen flow at 50° C., and the residue was dissolved in 50 μL of α(1-2,3,6)-mannosidase (15 U/mL) and zinc chloride (1 mM) in ammonium acetate buffer (0.05 M, pH 4.5) followed by incubation at 37° C. for 3 hours. The 2 μL of mixture was diluted with 18 μL of water followed by LC-MS/MS assay. The rest mixture was dried with nitrogen flow at 50° C., and the residue was dissolved in 50 μL of β-mannosidase (10 U/mL) in ammonium acetate buffer (0.1 M, pH 4.0) followed by incubation at 37° C. for 3 hours. The 2 μL of mixture was diluted with 18 μL of water followed by LC-MS/MS assay. The 2-AA-N-acetylglucosamine was prepared from reductive amination of N-acetylglucosamine in aqueous solution (10 μg/mL, 50 μL) with 2-AA and NaBH₃CN as described in profiling of oligosaccharides in human plasma, CSF, urine and cat CNS samples from GM1 gangliosidosis natural history study. LC-EPI scans of enzymatic degradation products were performed on a Shimadzu Prominence HPLC system coupled with the 6500QTRAP+ mass spectrometer. The ACE C18 column (4.6×150 mm, 3 μm) protected with a SecurityGuard C18 guard column (4×3 mm) was used to separate the degradation products from enzymatic reaction matrixes. The mobile phases consisted of 0.1% formic acid in water (solvent A) and 0.1% formic acid in methanol-acetonitrile (4:1) (solvent B), and the flow rate was 1 mL/min. The gradient was as follows: 0 to 8.4 min, 5 to 24% solvent B; 8.4 to 8.5 min, 24 to 100% solvent B; 8.5 to 10.5 min, 100% solvent B; 10.5 to 10.6 min, 100 to 5% solvent B; 10.6 to 12 min, 5% solvent B. The eluate was directed into the mass spectrometer for data acquisition from 4 to 10 min; elsewhere, eluate was sent to waste to minimize source contamination. The injection volume was 1 μL for all the degradation products, and the total run-time was 12 min. The ESI source temperature was 500° C.; the electrospray voltage was 5500 V; the DP was 75 V; the mass scan range was m/z 50-900; scan speed was 10000 u/s; and dynamic fill time was used; the CE for EPI scan of [M+H]⁺ ions of both 2-AA derivatized H3N2a and H3N2b digested with 1) β1,4 galactosidase (m/z 870.3); 2) β1,4 galactosidase, β-N-acetylglucosaminidase (m/z 667.2); 3) β1,4 galactosidase, β-N-acetylglucosaminidase, α-1-2,3,6-mannosidase (m/z 505.2); 4) β1,4 galactosidase, β-N-acetylglucosaminidase, α-1-2,3,6-mannosidase, and β-mannosidase (m/z 343.1), were 35, 35, 35, 25 eV, respectively. The collision and curtain gas were set at high and 30, respectively. Both desolvation gas and nebulizing gas were set at 35 L/min. Data were acquired and analyzed by Analyst software (version 1.6.3).

NaIO4 oxidation and NaBH4 reduction of 2-AA derivatized H3N2a and H3N2b: The HPLC fractions of 2-AA derivatized H3N2a and H3N2b were dissolved in 50 μL of NaIO₄ (40 mM) in ammonium acetate buffer (0.1 M, pH 6.5), and the reaction mixtures were left stand at 4° C. in the dark for 3 days. The oxidation was quenched by addition of 2 μL of ethylene glycol followed by standing at room temperature for 1 hour. To this reaction mixture was added 100 μL of NaBH₄ (10 mg/mL) in ammonium hydroxide (2 M) solution, the final reaction mixture was left stand at room temperature for 1 hour followed by quenching with 4.3 μL of acetic acid. The final mixture was directly submitted to LC-MS/MS assay.

LC-EPI scans of NaIO₄ and NaBH₄ treated products were performed on a Shimadzu Prominence HPLC system (Columbia, MD) coupled with the 6500QTRAP+ mass spectrometer (AB Sciex, Framingham, MA). The ACE C18 column (4.6×150 mm, 3 μm) protected with a SecurityGuard C18 guard column (4×3 mm) was used to separate the degradation products from enzymatic reaction matrixes. The mobile phases consisted of 0.1% formic acid in water (solvent A) and 0.1% formic acid in methanol-acetonitrile (4:1) (solvent B), and the flow rate was 1 mL/min. The gradient was as follows: 0 to 8.4 min, 5 to 36% solvent B; 8.4 to 8.5 min, 36 to 100% solvent B; 8.5 to 10.5 min, 100% solvent B; 10.5 to 10.6 min, 100 to 5% solvent B; 10.6 to 12 min, 5% solvent B. The eluate was directed into the mass spectrometer for data acquisition from 4 to 10 min; elsewhere, eluate was sent to waste to minimize source contamination. The injection volume was 20 μL for all the degradation products of 2-AA derivatized H3N2a and H3N2b, and the total run-time was 12 min. The ESI source temperature was 500° C.; the electrospray voltages were 5500 and −4500V for positive and negative modes, respectively; the DP was 75 and −75 V for positive and negative modes, respectively; the mass scan range was m/z 50-1000; scan speed was 10000 u/s; and dynamic fill time was used; the CEs for EPI scans of ions at m/z 948.4 (degradation product of 2-AA derivatized H3N2a, [M+H]⁺), m/z 976.4 (degradation product of 2-AA derivatized H3N2b, [M−H]⁺), m/z 946.4 (degradation product of 2-AA derivatized H3N2a, [M−H]⁻), m/z 974.4 (degradation product of 2-AA derivatized H3N2b, [M−H]⁻) were 50 50, −80, and −80 eV, respectively. The collision and curtain gas were set at high and 30, respectively. Both desolvation gas and nebulizing gas were set at 35 L/min. Data were acquired and analyzed by Analyst software (version 1.6.3).

### Synthesis of H3N2B and d6-H3N2B

Benzyl-3,4,6-O-tri-benzyl-α-D-mannopyranosyl-(1→3)-2-O-benzyl-4,6-O-benzylidene-β-D-mannopyranosyl-(1→4)-3,6-O-di-benzyl-2-deoxy-phthalimido-β-D-glucopyranoside (2) Compound 1 (10.68 g, 7.66 mmol, 1 eq) was dissolved in methanol/dichloromethane solvent mixture (3:2, 76 mL) followed by dropwise addition of sodium methoxide in methanol (0.5 M, 1.5 mL, 0.766 mmol, 0.1 eq). The reaction mixture was stirred overnight, quenched with ammonium chloride (0.65 g), sonicated for 5 min, and concentrated. The residue was purified by chromatography on a silica gel column (hexanes/ethyl acetate, 4:1→3:1→2:1→1:1) to give 2 (9.18 g, 88.5%). ESI-MS: [M+NH₄]⁺, calculated for C₈₂H₈₅N₂O₁₇⁺: m/z 1369.5843; found: m/z 1369.5812. ¹H NMR (CDCl₃, 400 MHz): δ 7.71-7.74 (m, 2H), 7.56-7.61 (m, 4H), 7.33-7.51 (m, 28H), 7.15-7.24 (m, 5H), 7.06-7.08 (m, 2H), 6.96-7.00 (m, 3H), 5.78 (brs, 1H), 5.65 (s, 1H), 5.49 (d, J=0.8 Hz, 1H), 5.29 (d, J=7.8 Hz, 1H), 4.92-5.06 (m, 5H), 4.84 (d, J=11.2 Hz, 1H), 4.80 (d, J=11.9 Hz, 1H), 4.77 (d, J=11.8 Hz, 1H), 4.75 (d, J=8.9 Hz, 1H), 4.51-4.67 (m, 6H), 4.41 (t, J=4.4 Hz, 2H), 4.33 (dd, J=10.4, 4.7 Hz, 1H), 3.88-4.24 (m, 8H), 3.80 (t, J=10.5 Hz, 2H), 3.58-3.69 (m, 5H); ¹³C NMR (CDCl3, 100 MHz): δ 167.9, 138.9, 138.7, 138.5, 138.3, 138.0, 137.9, 137.5, 137.3, 133.8, 131.8, 128.9, 128.8, 128.6, 128.5, 128.4, 128.3, 128.2, 128.1, 128.0, 127.9, 127.8, 127.1, 126.2, 123.3, 101.7, 101.2, 98.9, 97.6, 79.1, 78.8, 78.4, 78.1, 77.6, 75.7, 75.5, 75.2, 75.0, 74.6, 74.4, 73.7, 73.6, 72.5, 71.7, 70.9, 69.3, 68.5, 68.3, 67.1, 55.9.

Benzyl-O-(2,3,4,6-tetra-O-acetyl-β-D-galactopyranosyl)-(1→4)-O-(3,6-di-O-acetyl-2-deoxy-2-phthalimido-β-D-glucopyranosyl)-(1→2)-O-(3,4,6-tri-O-benzyl-α-D-mannopyranosyl)-(1→3)-O-(6-O-acetyl-2,4-di-O-benzyl-β-D-mannopyranosyl)-(1→4)-3,6-O-di-benzyl-2-deoxy-phthalimido-β-D-glucopyranoside (4) A mixture of compound 2 (8.1 g, 6 mmol, 1 eq) and lactosamine bromide 3 (19.2 g, 24 mmol, 4 eq) was azeotropically dried with toluene (2×100 mL), dissolve in dichoromethane (40 mL) and hexane (100 mL), evaporated, and then on high vacuum for 0.5 h. Freshly activated 4 Å molecular sieves (40 g) and dichoromethane (52 mL) were added, and the mixture was stirred for 0.5 hour under a nitrogen atmosphere in the dark. The mixture was cooled to −60° C., and 2,6-lutidine (2.79 mL, 24 mmol, 4 eq) was added. Silver trifluoromethanesulfonate (6.17 g, 24 mmol, 4 eq) was azeotropically dried with toluene (2×100 mL), then on high vacuum for 2 h, and dissolved in toluene (52 mL). The silver trifluoromethanesulfonate in toluene was added dropwise over 20 min to the mixture of compounds 2 and 3 and molecular sieves, and the final mixture was stirred and allowed to warm up to room temperature overnight. The reaction was quenched with saturated sodium thiosulfate solution. The mixture was diluted with dichloromethane and filtered through Celite. The organic phase is dried with magnesium sulfate, filtered, and concentrated in vacuum. The residue was purified by chromatography on a silica gel column (hexanes/ethyl acetate, 4:1→2:1→1:1) to give 4 (12.79 g, 92%). ESI-MS: [M+NH₄]⁺, calculated for C₁₁₄H₁₂₀N₃O₃₄⁺, m/z 2074.7748; found: m/z 2074.7707. ¹H NMR (CDCl3, 400 MHz): δ 7.70-7.73 (m, 2H), 7.46-7.67 (m, 11H), 7.22-7.41 (m, 20H), 7.12-7.21 (m, 5H), 7.01-7.12 (m, 5H), 6.91-6.98 (m, 2H), 6.82-6.92 (m, 3H), 5.66 (t, J=9.6 Hz, 1H), 5.46 (s, 1H), 5.41 (s, 1H), 5.24-5.32 (m, 1H), 4.99-5.23 (m, 4H), 4.65-4.97 (m, 7H), 4.41-4.61 (m, 5H), 4.21-4.41 (m, 5H), 3.74-4.21 (m, 14H), 3.36-3.72 (m, 9H), 2.78 (m, 2H), 2.17 (s, 3H), 2.15 (s, 3H), 2.08 (s, 3H), 2.02 (s, 3H), 1.97 (s, 3H); ¹³C NMR (CDCl3, 100 MHz): δ 169.9, 169.8, 169.7, 169.6, 169.4, 168.7, 167.5, 167.1, 138.3, 138.2, 137.9, 137.8, 137.3, 137.1, 131.4, 131.1, 131.0, 130.5, 128.8, 128.6, 128.5, 128.3, 128.9, 127.9, 127.8, 127.7, 127.6, 127.5, 127.5, 127.4, 127.3, 127.2, 127.2, 127.1, 126.5, 124.9, 123.5, 122.8, 101.8, 100.8, 100.4, 96.9, 94.7, 78.4, 77.7, 77.5, 76.3, 76.0, 74.5, 74.4, 74.1, 74.0, 73.5, 73.0, 72.4, 71.5, 71.3, 71.2, 70.8, 70.3, 70.1, 70.0, 69.9, 68.8, 68.2, 67.6, 66.3, 65.9, 60.9, 60.4, 55.2, 54.1, 21.1, 21.0, 20.2, 20.2, 20.1, 20.0.

O-β-D-galactopyranosyl-(1→4)-O-(2-acetamido-2-deoxy-β-D-glucopyranosyl)-(1→2)-O-α-D-mannopyranosyl-(1→3)-O-β-D-mannopyranosyl-(1→4)-2-acetamido-2-deoxy-α,β-D-glucopyranose (H3N2b) A mixture of 4 (7.63 g, 3.71 mmol), n-butanol (26 mL), and ethylenediamine (13 mL, 196 mmol, 53 eq) was heated at 90° C. for overnight. The mixture was evaporated in vacuum, and the residue was dissolved in methanol (15 mL) and co-evaporated with toluene (3×100 mL), and then on high vacuum for 2 days. The residue was then dissolved in pyridine (19.6 mL, 244 mmol, 66 eq) and acetic anhydride (19.6 mL, 196 mmol, 53 eq) was added and the mixture stirred overnight. The reaction was then concentrated under reduced pressure and the residue co-evaporated with toluene (2×10 mL) and dissolved in tetrahydrofuran (30 mL). Liquid ammonia (300 mL) was condensed at −78° C. into a 3-neck flask (1000 mL) with solid sodium (5 g, 219 mmol, 59 eq) equipped with mechanic stir and Dewar-type condenser. The resulting deep blue solution was stirred for 30 min. A solution of product from last step in tetrahydrofuran was added, and the reaction mixture was stirred for an additional 2 hours at −78° C. The reaction was quenched with methanol (50 mL). The reaction vessel was subsequently removed from its cooling bath and warmed to 25° C., and the ammonia was evaporated overnight. The mixture was dissolved in water (100 mL) and neutralized with 12N hydrochloride. The organic solvent in the mixture was removed by rotavapor. The aqueous solution of crude H3N2b was washed with tetrahydrofuran-ethyl acetate (2:1) (2×150 mL) and desalted by chromatography on a short charcoal column (water/acetonitrile, 1:0→3:2). The pure H3N2b (0.96 g, 28.4% overall yield over 3 steps) that is an anomeric mixture was obtained as white solid by isolation on a Hypercarb porous graphitic carbon HPLC column (4.6×150 mm, 3 μm) (Thermo Scientific, Waltham, MA) protected with a SecurityGuard C18 guard column (4×3 mm). The mobile phases consisted of 0.1% acetic acid in water (solvent A) and acetonitrile (solvent B), and the flow rate was 1 mL/min. The gradient was as follows: 0 to 15 min, 5 to 8% solvent B; 15 to 15.1 min, 8 to 10% solvent B; 15.1 to 18 min, 10% solvent B; 18 to 18.1 min, 10 to 95% solvent B; 18.1 to 21 min, 95% solvent B; 21 to 21.1 min, 95 to 5% solvent B; 21.1 to 25 min, 5% solvent B. ESI-MS: [M+H]⁺, calculated for C₃₄H₅₉N₂O₂₆⁺, m/z 911.3351; found: m/z 911.3327. [M+Na]⁺, calculated for C₃₄H₅₈N₂O₂₆Na⁺, m/z 933.3170; found: m/z 933.3145. ¹H NMR (D₂O, 600 MHz) β 5.20 (d, J=3 Hz, 0.64H), 5.13 (s, 1H), 4.71-4.75 (m, 1H), 4.58 (d, J=7.2 Hz, 1H), 4.47 (d, J=7.8 Hz, 1H), 4.25 (dd, J=3 Hz, 6.6 Hz, 1H), 4.20 (s, br, 1H), 3.98 (d, J=10.8 Hz, 1H), 3.89-4.96 (m, 6H), 3.83-4.96 (m, 2H), 3.46-3.82 (m, 20H), 2.06 (s, 3H), 2.05 (s, 3H). ¹³C NMR (D₂O, 150 MHz): 177.3, 177.0, 105.5, 102.6, 102.1, 102.1, 97.5, 93.1, 83.0, 82.0, 81.6, 81.1, 79.0, 78.7, 77.9, 77.3, 77.2, 76.1, 75.1, 74.9, 74.5, 73.5, 72.9, 72.9, 72.7, 72.0, 71.8, 71.1, 69.9, 68.5, 64.3, 63.6, 63.4, 62.7, 62.6, 62.6, 58.7, 57.4, 56.2, 24.9, 24.8, 24.5.

O-β-D-galactopyranosyl-(1→4)-O-(2-d₃-acetamido-2-deoxy-β-D-glucopyranosyl)-(1→2)-O-α-D-mannopyranosyl-(1→3)-O-β-D-mannopyranosyl-(1→4)-2-d₃-acetamido-2-deoxy-α,β-D-glucopyranose (d₆-H3N2b) The d₆-H3N2b (0.27 g, 20.4% overall yield over 3 steps) that is an anomeric mixture was prepared as white solid from 4 (2.96 g, 1.44 mmol) by reactions with: 1) n-butanol (10 mL), and ethylenediamine (5.1 mL, 76.3 mmol, 53 eq); 2) pyridine (7.6 mL, 95 mmol, 66 eq) and d₆-acetic anhydride (7.6 mL, 76.3 mmol, 53 eq); 3) liquid ammonia (120 mL) and sodium (1.95 g, 85 mmol, 59 eq) according to the procedures that was described for the preparation of H3N2b. ESI-MS: [M+H]⁺, calculated for C₃₄H₅₃D₆N₂O₂₆⁺, m/z 917.3727; found: m/z 917.3751. [M+Na]⁺, calculated for C₃₄H₅₂D₆N₂O₂₆Na⁺, m/z 939.3547; found: m/z 939.3572. ¹H NMR (D₂O, 600 MHz) δ 5.20 (d, J=3 Hz, 0.64H), 5.13 (s, 1H), 4.71-4.75 (m, 1H), 4.57 (d, J=7.2 Hz, 1H), 4.46 (d, J=7.8 Hz, 1H), 4.24 (dd, J=3 Hz, 6.6 Hz, 1H), 4.19 (s, br, 1H), 3.97 (d, J=10.8 Hz, 1H), 3.89-4.96 (m, 6H), 3.83-4.96 (m, 2H), 3.46-3.82 (m, 20H). ¹³C NMR (D₂O, 150 MHz) β 177.3, 177.1, 105.5, 102.6, 102.1, 102.0, 97.5, 93.1, 83.0, 82.0, 81.6, 81.0, 79.0, 78.7, 77.9, 77.3, 77.2, 76.1, 75.6, 75.1, 74.9, 74.5, 73.5, 72.9, 72.6, 72.0, 71.8, 71.1, 69.9, 68.5, 64.3, 63.6, 63.4, 62.7, 62.6, 62.5, 58.6, 57.4, 56.2.

CONFIRMATION OF H3N2B STRUCTURE: The endogenous and synthetic H3N2b were derivatized as described in profiling of oligosaccharides in human plasma, CSF, urine and cat CNS samples from GM1 gangliosidosis natural history study, and chromatographic separation was performed on ACE C18 column (150×4.6 mm, 3 μm) protected with a SecurityGuard C18 guard column (4×3 mm) using a Shimadzu Prominence HPLC system (Columbia, MD). The mobile phases consisted of 0.1% formic acid in water (solvent A) and 0.1% formic acid in methanol-acetonitrile (4:1) (solvent B), and the flow rate was 1 mL/min. The gradient was as follows: 0 to 20 min, 10 to 30% solvent B; 20 to 20.1 min, 30 to 100% solvent B; 20.1 to 22 min, 100 solvent B; 22 to 22.1 min, 100 to 10% solvent B; 22.1 to 24 min, 10 solvent B. The eluate was directed into the mass spectrometer for data acquisition from 5.0 to 22 min; elsewhere, eluate was sent to waste to minimize source contamination. The injection volume was 2 μL, and the total run-time was 24 min. The HPLC system was coupled to a Q-Exactive Orbitrap MS (Thermo Fisher Scientific, Waltham, MA) operating with a heated electrospray interface (HESI-II) in ESI. The vaporizer temperature, the capillary temperature, spray voltage, sheath gas flow rate, auxiliary gas flow rate, ion sweep gas pressure, and S-lens RF level were set to 300° C., 270° C., 3.5 kV, 60, 30, 2, and 55, respectively. The parallel reaction monitoring scan of m/z 1032.4 was acquired at a resolution of 70,000 and normalized collision energy of 30% in positive ion modes. Ion accumulation was set at 3.0×10⁶ of Automatic Gain Control (AGC) and a maximum injection time of 100 ms. The analyses were performed without lock mass. Data were processed using Xcalibur™ version 2.2.1 (Thermo Fisher Scientific, Waltham, MA).

VALIDATION OF METHODS FOR H3N2B IN HUMAN URINE, PLASMA, AND CSF: Stock solution preparation—The stock solutions of H3N2b (1 mg/mL) and d₆-H3N2b (1 mg/mL) were prepared in water. The internal standard working solution (1 μg/mL of d₆-H3N2b) for human urine, plasma, and CSF was prepared in methanol-water (4:1).

Standard curve and quality control (QC) samples—Because of the endogenous presence of H3N2b in human plasma, the calibration standards for urine (10, 20, 40, 200, 1000, 2000, 5000, 10000 ng/mL), plasma (1, 2, 5, 10, 50, 100, 250, 500 ng/mL), and CSF (1, 2, 5, 10, 50, 100, 250, 500 ng/mL) were prepared by spiking the H3N2b into water and serial dilutions, and the water was used as blank. The pooled human urine, plasma, and CSF were analyzed to establish the mean concentration of endogenous H3N2b levels and used to prepare low QC (LQC), middle (MQC), high QC (HQC), and dilution QC (DQC). The LQC (endogenous level+100 ng/mL for urine; endogenous level+10 ng/mL for plasma, endogenous level+10 ng/mL for CSF), MQC (endogenous level+4000 ng/mL for urine; endogenous level+200 ng/mL for plasma, endogenous level+200 ng/mL for CSF), HQC (endogenous level+8000 ng/mL for urine; endogenous level+400 ng/mL for plasma, endogenous level+400 ng/mL for CSF), and DQC (endogenous level+16000 ng/mL for urine; endogenous level+800 ng/mL for plasma; endogenous level+800 ng/mL for CSF) were prepared. The H3N2b in the DQC samples were higher than the upper limit of quantification (ULOQ), and the DQC samples were diluted 1:4 with water, prior to extraction.

Sample preparation—For urine, standards, QCs, blank or study samples (50 μL) were aliquoted into 2 mL polypropylene tubes. To each sample 50 μL of internal standard (1 μg/mL of d₆-H3N2b in methanol-water (4:1) was added and 50 μL of methanol-water (4:1) was used for a blank followed by addition of 200 μL of methanol. The samples were vortexed for 3 min, centrifuged for 10 min at 9400 g, and supernatant transferred and dried with nitrogen flow at 50° C. into clean 1.2 mL glass inserts. To each insert 2-AA (30 mg/mL), NaBH₃CN (20 mg/mL), sodium acetate trihydrate (40 mg/mL) and boric acid (20 mg/mL) in methanol-water (4:1) (50 μL) was added. The mixtures were vortexed for 3 min, heated at 50° C. for 1 hour, and dried with nitrogen flow at 50° C. The residues were partitioned between water (200 μL) and MTBE (600 μL), and aqueous phases were transfer to new 1.2 mL glass inserts for LC-MS/MS assay.

For plasma and CSF, standards, QCs, blank or study samples (100 μL) were aliquoted into 2 mL polypropylene tubes. To each sample 50 μL of internal standard (1 μg/mL of d₆-H3N2b in methanol-water (4:1) was added and 50 μL of methanol-water (4:1) was used for a blank followed by addition of 400 μL of methanol. The samples were vortexed for 3 min, centrifuged for 10 min at 9400 g, and supernatant transferred and dried with nitrogen flow at 50° C. into clean 1.2 mL glass inserts. To each insert 2-AA (30 mg/mL), NaBH₃CN (20 mg/mL), sodium acetate trihydrate (40 mg/mL) and boric acid (20 mg/mL) in methanol-water (4:1) (50 μL) was added. The mixtures were vortexed for 3 min, heated at 50° C. for 1 hour, and dried with nitrogen flow at 50° C. The residues were partitioned between water (200 μL) and MTBE (600 μL), and aqueous phases were transfer to new 1.2 mL glass inserts for LC-MS/MS assay.

LC-MS/MS analysis—LC-MS/MS analysis was conducted on a Shimadzu Prominence HPLC system coupled with the 6500QTRAP+ mass spectrometer. The H3N2b and d₆-H3N2b were separated on a Halo Phenyl-Hexyl (4.6 mm×150 mm, 2.7 μm) (Mac-Mod Analytical, Chadds Ford) protected with a SecurityGuard Phenyl guard column (4×3 mm) (Phenomenex, Torrance, CA). The mobile phases consisted of 0.1% acetic acid in water (solvent A) and methanol-acetonitrile (1:1) (solvent B), and the flow rate was 0.8 mL/min. The gradient was as follows: 0 to 15 min, 10 to 20% solvent B; 15 to 15.1 min, 20 to 100% solvent B; 15.1 to 17 min, 100% solvent B; 17 to 17.1 min, 100 to 10% solvent B; 17.1 to 19 min, 10% solvent B. The eluate was directed into the mass spectrometer for data acquisition from 11.5 to 14.5 min. The total run-time was 19 min, and the injection volume was 5, 10, 10 μL for urine, plasma, and CSF samples, respectively. The ESI source temperature was 500° C.; the electrospray voltage was 5500 V; the DP, EP and the CXP was 40, 10 and 10 V, respectively. The collision and curtain gas were set at medium and 30, respectively. The desolvation gas and nebulizing gas were set at 50 and 60 L/min, respectively. The [M+2H]²⁺ ion of H3N2b was used to set up the MRM transitions, which showed higher sensitivity than [M+H]⁺ ion. The CEs for MRM transitions of m/z 516.7 to 667.3 (quantifier for 2-AA derivatized H3N2b), m/z 516.7 to 343.1 (qualifier for 2-AA derivatized H3N2b), and m/z 519.7 to 670.3 (d₆-H3N2b) were 15, 15, and 15 eV, respectively. The dwell time was set at 100 ms for all the MRM transitions. Data were acquired and analyzed by Analyst software (version 1.6.3). Calibration curves were constructed by plotting the corresponding peak area ratios of analyte/internal standard versus the corresponding analyte concentrations using weighted (1/x²) linear least squares regression analysis.

Validation of linearity, precision and accuracy—The linearity, precision and accuracy of assay were evaluated over three analytical runs. In the validation analytical run, a set of samples that were analyzed in one batch, including calibration standards in duplicate, a blank, a blank with internal standard, and QC samples. This set of samples were prepared and analyzed in three different batches. The replication was the repeated preparation and analysis of the same sample. The linearity response of H3N2b was assessed over 10-10000 ng/mL, 1-500 ng/mL, and 1-500 ng/mL, respectively for urine, plasma, and CSF. The precision and accuracy of the assay were determined at LLOQ, LQC, MQC and HQC levels. For each QC concentration, analysis was performed in six replicates on each day except for DQCs for which three replicates were prepared in the first batch. DQC samples were analyzed in three replicates. Accuracy and precision were denoted by percent relative error (% RE) and percent coefficient of variance (% CV), respectively. The accuracy and precision were required to be within ±15% RE of the nominal concentration and <15% CV, respectively, for LQC, MQC, HQC, DQC samples. The accuracy and precision were required to be within ±20% RE of the nominal concentration and <20% CV for LLOQ samples in the intra-batch and inter-batch assays.

Validation of selectivity and specificity—To ascertain the selectivity of the method, blank and blank with internal standard, an ULOQ calibrator without the internal standard, and six different lots of blank biological samples (human plasma, CSF, and urine) were analyzed. To evaluate the specificity, commercially available N-complex glycans, including A1F glycan, A3 glycan, NA2F glycan, NA2 glycan, which are precursors of H3N2b, were evaluated for interferences to H3N2b. For double blank, any response at the retention times of the H3N2b was to be <20% of the response of the LLOQ, and any response at the retention time of the internal standard was to be <5% of the mean response of the internal standard in the calibration curve. For the ULOQ calibrator without internal standard, any response at the retention time of the internal standard was to be <5% of the mean response of the internal standard in the calibration curve. For the blank with internal standard, any response at the retention times of the H3N2b is to be <20% of the response of the LLOQ. For the blank biological samples, any response at the retention time of the internal standard was to be <5% of the mean response of the internal standard in the calibration curve. The interferences to H3N2b in N-complex glycans should be <20% of the response of the LLOQ.

Validation of stability—The long-term storage, freeze/thaw stabilities, and stabilities on the bench-top and in the autosampler were determined at the LQC and HQC concentration levels (n=3). Long-term storage stabilities of H3N2b—80° C. in urine, plasma, CSF were tested. Bench-top stabilities of H3N2b were evaluated in urine at room temperature, in plasma at room temperature, in CSF at room temperature and 4° C. Freeze/thaw stability in urine, plasma, CSF was tested by freezing the samples at −80° C. overnight, followed by thawing to room temperature the next day. This process was repeated three times. In the autosampler, stabilities of processed urine, plasma, CSF samples at 4° C. were tested over 3 days by injecting the first batch of the validation samples. Stock solution stability was established by quantification of samples from dilution of two stock solutions that were stored at −20° C. and at room temperature on the bench, respectively, to the final solution (10000 ng/mL in water). The stabilities of H3N2b in the matrix of standard curve for urine, plasma, CSF were test after storage at room temperature and at −80° C. Afresh standard curve was established each time.

Validation of stability—Carryover was assessed by injecting a blank sample immediately after an injection of the ULOQ calibrator.

Validation Results: Linearity and parallelism—The standard curve linear ranges for urine, plasma, and CSF were 10-10000 ng/mL, 1-500 ng/mL, 1-500 ng/mL, respectively. The r values of standard curves for urine, plasma, and CSF were >0.99, and the difference in slopes of standard curves between surrogate and biological matrixes were <6.2%. The accuracies of calibrators of standard curves for urine, plasma, and CSF were within 85-115%.

Sensitivity—The method sensitivities were defined by the lower limits of quantification (LLOQ) which were 10 ng/mL, 1 ng/mL, 1 ng/mL for urine, plasma, and CSF, respectively. The sensitivity test showed that the intra- and inter-batch accuracies of the LLOQ samples were within ±10% relative error (RE), and the intra- and inter-batch coefficients of variation (CV) of the LLOQ samples were <15% for urine, plasma, and CSF methods.

Selectivity—The selectivity test showed that 1) the response in blank at the retention times of the H3N2b and internal standard were <20% of the response of the LLOQ and <5% of the mean response of the internal standard in the calibration curve, respectively; 2) the responses in the upper limit of quantification (ULOQ) calibrator without internal standard at the retention time of the internal standard were <5% of the mean response of the internal standard in the calibration curve; 3) the responses in the blank with internal standard at the retention times of the H3N2b were <20% of the response of the LLOQ; 4) the responses in blank biological samples at the retention time of the internal standard were <5% of the mean response of the internal standard in the calibration curve. No interferences to H3N2b in N-complex glycans were observed in specificity test.

Accuracy and precision—The method accuracies and precisions were evaluated with quality control (QC) samples at low (LQC), middle (MQC), and high (HQC) levels, and the urine, plasma, and CSF methods showed intra- and inter-batch accuracies and precisions within ±15% RE and <10% CV, respectively.

Stability—The H3N2b was stable at room temperature in urine for 24 hr, after 3 freeze/thaw cycles, for 3 days in processed urine samples at 4° C., and for 194 days at −80° C. The H3N2b was stable at room temperature in plasma for 24 hr, after 3 freeze/thaw cycles, for 3 days in processed plasma samples at 4° C., and for 158 days at −80° C. The H3N2b was stable at room temperature in CSF for 2 hr and at 4° C. for 24 hr, after 3 freeze/thaw cycles, for 3 days in processed CSF samples at 4° C., and for 125 days at −80° C. The H3N2b in stock solution was stable at room temperature for 24 hr and −20° C. for 232 days. The H3N2b in the matrix of standard curve for urine, plasma, CSF was stable at room temperature for 24 hr. The standard curve samples for urine, plasma, and CSF were stable at −80° C. for 194, 158, and 125 days, respectively. The stock solution of H3N2b was stable at −20° C. for 231 days.

Carryover—No carryovers were observed in blank samples following the ULOQ samples for urine, plasma, and CSF.

Dilution integrity—The dilution integrity was evaluated with dilution QC (DQC) samples, and accuracy (within ±10% RE) and precision (<5% CV) were observed after 5-fold dilution of urine, plasma, and CSF samples.

### Example 2: Pentasaccharides as GM1-Gangliosidosis Biomarkers for Diagnosis and Assessment of Disease Severity and Therapeutic Efficacy

Although gene therapy has shown great promise in a cat model of GM1-gangliosidosis by delaying symptom onset, reducing lysosomal storage in the brain and peripheral tissues, and prolonging lifespan, evaluation of its treatment efficacy in clinical trials is challenging due to the small and heterogeneous patient population as well as slow progression in the non-infantile patients. Recently, a biomarker was discovered that is markedly elevated in biofluids from GM1-gangliosidosis patients and in the central nervous system (CNS) of the cat model and was reduced significantly in the CNS of the cat model in response to gene therapy, and thus the biomarker has potential as a pharmacodynamics/response biomarker. In this example, clinical assays were developed for the biomarker in human urine, plasma, and CSF, and use these assays for assessing the efficacy of gene therapy in clinical trials.

Evaluation of treatment efficacy with clinical endpoints is challenging due to the small and heterogeneous patient population as well as slow progression in the non-infantile patients. Therefore, the use of validated biomarkers as surrogate outcome measures to predict clinical benefits is critical to accelerate drug development. A wide range of potential biomarkers have been explored in the GM1-gangliosidosis cat model to assess clinical efficacy. Cerebrospinal fluid (CSF) ganglioside GM1 appeared the most promising exhibiting >30-fold elevation in untreated GM1-gangliosidosis cats, which was reduced in response to AAV-treatment; however, ganglioside GM1 is elevated less than 2-fold in the CSF and plasma of GM1-gangliosidosis patients. Several oligosaccharides have been found to be significantly elevated in urine from GM1-gangliosidosis patients and used for diagnosis of this disorder. In preliminary studies, oligosaccharides were profiled with liquid chromatography-tandem mass spectrometry (LC-MS/MS) in samples from GM1-gangliosidosis patients and the cat model, confirming the previous findings. A pentasaccharide, O-β-D-galactopyranosyl-(1→4)-O-(2-acetamido-2-deoxy-β-D-glucopyranosyl)-(1→2)-O-α-D-mannopyranosyl-(1→3)-O-β-D-mannopyranosyl-(1→4)-2-acetamido-2-deoxy-α,β-D-glucopyranose, which is referred to as H3N2b, was elevated >20-fold in patient urine, plasma, and CSF, and in the central nervous system (CNS) of the GM1-gangliosidosis cats. The CNS H3N2b levels in the GM1-gangliosidosis cats were reduced in response to AVV-treatment. H3N2b is a natural substrate of β-galactosidase, and its level is negatively correlated with enzyme activity. The H3N2b oligosaccharide can be used as a pharmacodynamics/response biomarker for treatments that restore or replace β-galactosidase activity. Application of validated methods is critical to reliably and accurately measure H3N2b, and thus provides necessary data to support the treatment effectiveness. The present example validated LC-MS/MS methods for determination of H3N2b in human urine, plasma, CSF, which are useful to assess AVV treatment efficacy in Phase 1/2 clinical trial.

The major storage products of GM1-gangliosidosis include three types of β-galactosidase natural substrates: glycosphingolipids, oligosaccharides, and keratan sulfate. Ganglioside GM1 is the main storage product in brain, and to a much lesser extent, its asialo-derivative GA1. The level of ganglioside GM1 stored in the brain does not differ significantly between infantile and late infantile/juvenile patients. In adult/chronic cases, ganglioside GM1 is elevated only in cerebral cortex and white matter. Water-soluble mucopolysaccharides were detected in large amounts in liver and spleen of GM1-gangliosidosis patients. These storage products were identified as keratan sulfate on the basis of its composition and electrophoretic mobility. Heterogeneous galactose-containing oligosaccharides, which are derived from N-glycoproteins, are stored in liver and excreted in urine of GM1-gangliosidosis patients of all clinical types. The accumulation of high molecular weight oligosaccharides was reported to increase progressively with the severity of clinical features. However, the semi-quantification with readouts of “detected” and “not detected” in this report did not permit correlation of easily detectable low molecular weight oligosaccharides with the severity of clinical features.

Inflammatory responses are considered to contribute to the pathogenesis or disease progression in GM1-gangliosidosis. Inflammatory mediators in CSF such as heparin-binding EGF-like growth factor, Tamm-Horsfall urinary glycoprotein, α-1-antichymotrypsin, macrophage migration inhibitory factor, sex hormone-binding globulin, matrix metalloproteinase-3 are elevated in infantile and late-infantile GM1-gangliosidosis, while epithelial-derived neutrophil-activating protein 78, monocyte chemotactic protein 1, macrophage inflammatory protein-1α, macrophage inflammatory protein-1β, tumor necrosis factor receptor 2 are elevated only in infantile patients. Elevations of these markers, however, are modest, and there is significant overlap between control and GM1-gangliosidosis.

Magnetic resonance imaging (MRI) showed that degree of brain atrophy worsened over time and correlated with the decline in clinical metrics, language, and ambulation, and magnetic resonance spectroscopy (MRS) demonstrated a deficit of N-acetylaspartate in brains of both the late infantile and juvenile patients with greater in the late infantile patients. Electroencephalogram (EEG) alterations are mild in early-stage GM1 gangliosidosis patients and deteriorate suddenly after reaching a critical threshold of pathology. The CSF aspartate aminotransferase (AST) and lactate dehydrogenase (LDH) are moderately elevated in GM1-gangliosidsosis cats and human late-infantile patients and correlated with the severity of clinical neurologic disease. On the other hand, serum LDH from GM1-gangliosidosis cats and patients are not elevated, and serum AST is only elevated in late-infantile, but not juvenile patients, and decreased over time.

Although enzyme analysis of the β-galactosidase can be used to assess AAV-treatment efficacy, the use of artificial substrates in the in vitro enzyme assay may not exactly replicate in vivo enzyme activity with natural substrates. There were reports that β-galactosidase testing was reportedly normal in GM1-gangliosidosis patients. A wide range of biomarkers have been explored in the GM1-gangliosidosis cat model to assess clinical efficacy, including CSF sphingolipids and AST; blood calcium, alkaline phosphatase, alanine aminotransferase, and inflammatory marker globulin; urine glycosaminoglycan (keratan sulfate); and EEG, brain MRI, and MRS. Of these, CSF ganglioside GM1 appeared the most promising exhibiting >30-fold elevation in untreated GM1-gangliosidosis cats, which was reduced in response to AAV gene therapy treatment; however, ganglioside GM1 is elevated less than 2-fold in the CSF and plasma of GM1-gangliosidosis patients. The urine keratan sulfate was elevated 6-fold in GM1-gangliosidosis cat and reduced after AAV treatment. We further examined whether oligosaccharides could be used to assess AVV treatment efficacy in cat CNS. A pentasaccharide (O-β-D-galactopyranosyl-(1→4)-O-(2-acetamido-2-deoxy-β-D-glucopyranosyl)-(1→2)-O-α-D-mannopyranosyl-(1→3)-O-β-D-mannopyranosyl-(1→4)-2-acetamido-2-deoxy-α,β-D-glucopyranose), which is referred to as H3N2b, was elevated >100-fold in brain stem, cerebellum, cervical intumescence, frontal cortex, lumbar intumescence, occipital cortex, parietal cortex, temporal cortex, and thalamus. H3N2b was significantly reduced after AAV-treatment in all regions except for brain stem, cervical intumescence, and lumbar intumescence, which were difficult to infect by intracranial injection of AAV. The H3N2b and its isomer H3N2a (O-β-D-galactopyranosyl-(1→4)-O-(2-acetamido-2-deoxy-β-D-glucopyranosyl)-(1→2)-O-α-D-mannopyranosyl-(1→6)-O-β-D-mannopyranosyl-(1→4)-2-acetamido-2-deoxy-α,β-D-glucopyranose) were elevated >20-fold in urine, plasma, CSF from GM1-gangliosidosis patients, but H3N2a was undetectable in cat CNS samples.

H3N2b was selected for further development as a pharmacodynamics/response biomarker for treatment targeted at augmenting β-galactosidase activity because it is present in both the cat model and patients, thus facilitating translation of treatment developed in the cat model to humans. H3N2b is a natural substrate of β-galactosidase that plays a central role in GM1-gangliosidosis, thus its level is inversely proportional to enzyme activity. Unlike other GM1-gangliosidosis biomarkers that can only serve as either peripheral or CNS biomarker, H3N2b can assess the treatment efficacy in both CNS and visceral organs. In addition, H3N2b provides large dynamic range to evaluate the treatment efficacy. Moreover, H3N2b is easy to measure serially in biofluids such as urine, plasma, and CSF, making it appropriate as monitoring biomarker. In particular, the response of the biomarker to AAV gene therapy indicates that it is a suitable pharmacodynamics/response biomarker for treatment targeted at β-galactosidase activity.

H3N2b in urine, plasma and CSF can serve as pharmacodynamics/response biomarker in the development of treatment for GM1-gangliosidosis that restores or replaces enzymatic activity. It can provide information about: 1) whether the enzymatic activity is increased after treatment; 2) to what degree the enzymatic activity is increased; 3) whether the enzymatic activity is increased in peripheral organs and CNS; and 4) selection of optimal dosing and administration route. In this project, we will use H3N2b to assess the efficacy of AAV-treatment in Phase 1/2 trial.

Several analytical methods have been developed to profile the oligosaccharides in GM1-gangliosidosis, including thin layer chromatography (TLC), high performance liquid chromatography (HPLC) with radio detection or pulsed amperometric detection (PAD), matrix-assisted laser desorption ionization (MALDI) mass spectrometry, electrospray ionization-tandem mass spectrometry (ESI-MS/MS), high-pH anion-exchange chromatography(HPAEC)-PAD, HPAEC-electrospray ionization mass spectrometry(ESI-MS), liquid chromatography-tandem mass spectrometry (LC-MS/MS). The oligosaccharides were detected as both derivatized and native forms. Chromatography is necessary to separate the H3N2a and H3N2b; however, no reported methods can separate H3N2a and H3N2b from other two interfering isomeric pentasaccharides. All the methods reported so far provided only semi-quantification due to lack of standard compounds.

A LC-MS/MS method was developed to detect H3N2b, in which H3N2b is derivatized with 2-aminobenzoic acid (2-AA) to improve the mass spectrometric detection sensitivity of oligosaccharides and separation of oligosaccharide isomers. This method can baseline separate H3N2b from all isobaric interferences, and paved a way for accurate quantification of H3N2b. A simple and high throughput sample preparation was developed that involves protein precipitation and 2-AA derivatization in one step. H3N2b and its deuterated internal standard d₆-H3N2b were synthesized allowing method development and validation according to FDA guidance for bioanalytical method validation and fit-for-purpose validation principle.

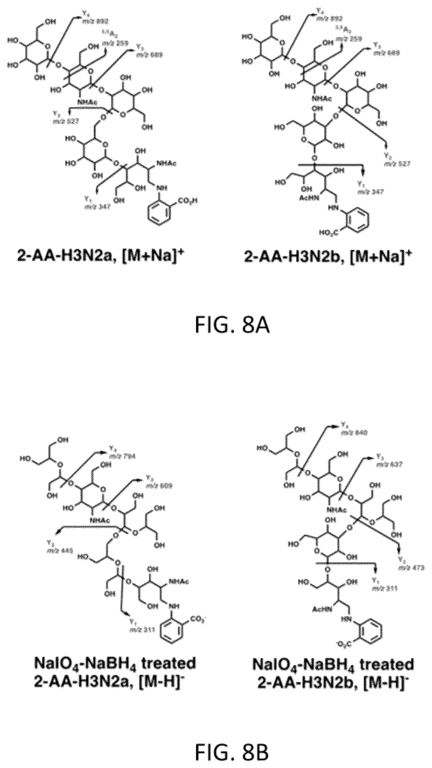

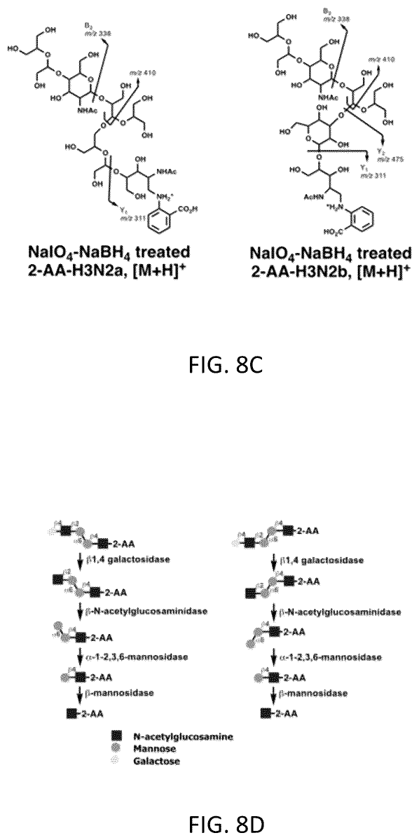

The H3N2a and H3N2b oligosaccharides were identified in urine from patients with GM1-gangliosidosis disease and Morquio B syndrome, and characterized as underivatized- and permethylated-oligosaccharides on different MS platforms. Only the structure of H3N2a isolated from urine of a Morquio B patient was confirmed with ¹HNMR. The H3N2a and H3N2b were derivatized with 2-AA in this example to improve the mass spectrometric sensitivity and separation from other isomers, and the structural information obtained from underivatized- and permethylated-forms is different from 2-AA derivatives. There are unknown interfering isomers (peak 1 and 3) of H3N2a and H3N2b in human samples, which have not been reported previously. Rigorous structure characterization was performed to identify the structures of H3N2a and H3N2b, including tandem mass spectrometry, chemical and enzymatic degradations. The 2-AA derivatized H3N2a and H3N2b can be detected as [M+H]⁺, [M−H]⁻, and [M+Na]⁺ ions, which generated unique product ions in collision-induced dissociation (CID). The Y1-Y4 ions generated from glycosidic bond cleavages of [M+H]⁺ (m/z 343, 505, 667, 870), [M+Na]⁺ ions (m/z 365, 527, 689, 892) (FIG. 8A), and [M−H]− (m/z 341, 503, 665, 868) (data not shown) indicate that the sequences of both oligosaccharides are ⁵Hexose-⁴N-acetylhexosamine-³Hexose-²Hexose-¹N-acetylhexosamine. The cross-ring cleavage of ^{3,5}A₂ (m/z 259) in [M+Na]⁺ ions of 2-AA-H3N2a and 2-AA-H3N2b suggest that ⁵Hexose-⁴N-acetylhexosamine is connected by 1,4-linkage (FIG. 8A). Periodate (NaIO₄) oxidation can selectively cleave adjacent hydroxyl groups to the aldehydes that are converted into more stable alcohols by sodium borohydride (NaBH₄) reduction. The CID of [M−H]− ion of product from NaIO₄ oxidation—NaBH₄ reduction of 2-AA-H3N2a shows open of pyranose ring without loss of carbon in ³Hexose (Y3 ion at m/z 609), losses of CHOH from ²Hexose (Y₂ ion at m/z 445) and CH₂OH from ¹N-acetylhexosamine (Y₁ ion at m/z 311), suggesting that ⁴N-acetylhexosamine-³Hexose, ³Hexose-²Hexose and ²Hexose-¹N-acetylhexosamine are connected by 1,2 (or 1,3)-, 1,6- and 1,4-linkages, respectively (FIG. 8B). Fragmentation of [M−H]⁻ ion of NaIO₄—NaBH₄ treated 2-AA-H3N2b demonstrates open of pyranose ring without loss of carbon in ³Hexose (Y3 ion at m/z 637), intact ²Hexose (Y2 ion at m/z 473), and loss of CH₂OH from ¹N-acetylhexosamine (Y1 ion at m/z 311), suggesting that 4N-acetylhexosamine-³Hexose, ³Hexose-²Hexose and ²Hexose-¹N-acetylhexosamine are connected by 1,2 (or 1,3)-, 1,3- and 1,4-linkages, respectively (FIG. 8B). The product ion at m/z 410 from CID of [M+H]⁺ ions of NaIO₄—NaBH₄ treated 2-AA-H3N2a and 2-AA-H3N2b suggests that their 4N-acetylhexosamine-³Hexose is connected by 1,2-linkage (FIG. 8C). The sequential loss of galactose, N-acetylglucosamine, mannose, mannose from 2-AA-H3N2a and 2-AA-H3N2b was observed following the stepwise digestion of H3N2b with β1,4 galactosidase, β-N-acetylglucosaminidase, α-1-2,3,6-mannosidase, and β-mannosidase (FIG. 8D). The reducing terminus of both 2-AA-H3N2a and 2-AA-H3N2b is 2-AA-N-acetylglucosamine that was confirmed with standard compound. The H3N2a and H3N2b were identified as O-β-D-galactopyranosyl-(1→4)-O-(2-acetamido-2-deoxy-β-D-glucopyranosyl)-(1→2)-O-α-D-mannopyranosyl-(1→6)-O-β-D-mannopyranosyl-(1→4)-2-acetamido-2-deoxy-α,β-D-glucopyranose and O-β-D-galactopyranosyl-(1→4)-O-(2-acetamido-2-deoxy-β-D-glucopyranosyl)-(1→2)-O-α-D-mannopyranosyl-(1→3)-O-β-D-mannopyranosyl-(1→4)-2-acetamido-2-deoxy-α,β-D-glucopyranose, respectively. They are moieties of complex N-glycan.

A simple and sensitive LC-MS/MS method was developed to detect H3N2b in plasma, urine and CSF, in which reductive amination of H3N2b with 2-AA and sodium cyanoborohydride (NaBHCN) is used to increase its hydrophobicity and subsequently retention on reversed phase HPLC column. The extraction of H3N2b from plasma, urine and CSF and 2-AA derivatization were performed in one step (i.e., plasma, urine and CSF was mixed with derivatization reagents in methanol), the mixture was centrifuged to remove precipitated protein after heating at 80° C. for 1 hour, the excess 2-AA and hydrophobic lipids were removed after supernatant was dried and partitioned between water and methyl tert-butyl ether (MTBE), and the aqueous phase was directly injected to LC-MS/MS system. Chromatographic separation was achieved on an ACE C18 column (4.6×150 mm). Detection was performed on an AB-Sciex 6500QTRAP+ mass spectrometer operated in positive electrospray multiple reaction monitoring (MRM) mode.

Hemiacetals in oligosaccharides are masked aldehydes, and because aldehydes are rare functionalities in cells, reductive amination with 2-AA is selective for oligosaccharides. The mass spectrometric sensitivity for H3N2b was significantly improved due to introduction of a readily ionizable amino group, and allowed detection of previously undetectable plasma and CSF H3N2b. The reductive amination of H3N2b with 2-AA also eliminates α- and β-anomers, and 2-AA-H3N2b appear on chromatograms as a single peak, rather than two α- and β-anomer peaks. More importantly the 2-AA-H3N2b was baseline separated from other isomeric interferences, which is critical to meet the requirement of method selectivity/specificity. Because minimal sample transfer was used in extraction and derivatization, the H3N2b loss during the sample preparation was minimized. The reductive amination condition was carefully optimized to achieve quantitative yield under neutral condition, which minimizes the decomposition of H3N2b.

As no commercial reference standard of H3N2b and its stable isotope-labeled analog as internal standard are available, H3N2b and d₆-H3N2b were synthesized. The d₆-H3N2b as internal standard is essential for a robust LC-MS/MS method as it can compensate for fluctuations in extraction, derivatization, ionization efficiency, and mass spectrometric performance. Glycosylation of acceptor 1 and donor 2 will afford protected pentasaccharide 3. Removal of the N-phthaloyl and O-acetyl protective groups and acetylation with acetic anhydride and d₆-acetic anhydride followed by complete deprotection will furnish H3N2b and d₆-H3N2b, respectively (Scheme 1). The final products of H3N2b and d₆-H3N2b were purified with preparative HPLC to achieve >95% purity.

After synthesizing the H3N2b and d₅-H3N2b, the LC-MS/MS method for H3N2b was further optimized, focusing on stock solution stability, optimization of mass spectrometric parameters, further optimization of LC condition, standard curve matrix selection, linear ranges of standard curves for human plasma, urine, and CSF, matrix effect, recovery, stabilities of benchtop and freeze/thaw H3N2b in biological matrixes, and carryover. The stability of aqueous H3N2b stock solution in glassware and plasticware tested to ensure no absorption loss. The mass spectrometric parameters for 2-AA-H3N2b further optimized to reach optimal sensitivity. H3N2a is an endogenous compound, and analyte-free biological matrices are not available. Water and buffers evaluated as surrogate matrix for standard curve, in which H3N2b is stable and there are no interferences. As the H3N2b levels in plasma, urine, and CSF are different, the linear ranges of standard curves for plasma, urine, and CSF determined to cover endogenous levels of H3N2b in most of biological samples. The volume ratio of methanol to biological sample optimized to achieve close and consistent recoveries of H3N2b from biological and surrogate matrixes. The storage condition of biological matrixes on benchtop and thaw condition optimized to ensure no H3N2b is decomposed. The LC condition optimized to shorten the run time and eliminate the matrix effect. The needle washing solvent and LC gradient optimized to eliminate carryover. The standard operation procedures (SOP) established.

Sample collection, storage and shipment: The plasma, urine, and CSF samples collected on day −20, and at 3, 6, 12, and 18 months, and at years 2, 3, 4, and 5 from AAV-treated patients enrolled in the Phase 1/2 trail (Dr. Tifft at NHGRI). The 10 mL of blood collected in EDTAK2 tube and centrifuged at 4° C. and 1500 g for 10 minutes. Centrifugation performed within 30 minutes after blood is drawn. The plasma transferred and aliquoted into polypropylene tubes (1 mL/tube). The urine sample collected from the middle of the urine flow into a urine collection cup, and aliquoted into polypropylene tubes (1 mL/tube). The CSF sample collected via lumbar puncture in the space between the bones (vertebrae) in the lower back, and about 12 mL of fluid collected through the needle under anesthesia. The CSF sample aliquoted into polypropylene tubes (1 mL/tube). All the samples will be stored in −80° C. freezer until being shipped to analytical lab with dry ice.

Sample analysis: The validated method for H3N2b used to analyze clinical samples. Samples consisting of calibration standards in duplicate, a double blank, a blank, QC standards (LQC, MQC and HQC), and unknown study samples analyzed. The standard curve covered the expected unknown sample concentration range, and samples that exceeded the highest standard diluted and re-assayed. In the dilution sample re-assay, a diluted QC in triplicate included in the analytical run. The results of the QC samples provide the basis of accepting or rejecting the run based on at least 67% ( 4/6) of the QCs fall within 15% of their nominal values according to FDA guidelines.

What is claimed is:

1. A method for detecting GM1-gangliosidosis in a subject having or suspected of having symptoms of GM1-gangliosidosis, the method comprising:
(a) measuring pentasaccharide levels, in a urine, a blood sample or a CSF sample obtained from the subject; and
(b) using the measurements of (a) to classify the subject as having GM1-gangliosidosis.

2. A method for detecting GM1-gangliosidosis severity in a subject having or suspected of having symptoms of GM1-gangliosidosis, the method comprising:
(a) measuring pentasaccharide levels, in a urine, a blood sample or a CSF sample obtained from the subject; and
(b) using the measurements of (a) to classify the severity GM1-gangliosidosis.

3. The method of claim 1 or claim 2, wherein measuring pentasaccharide levels includes processing the urine, the blood sample or the CSF sample from the subject comprising the steps of adding an internal standard, depleting one or more protein(s) and derivatizing one or more pentasaccharides.

4. The method of claim 3, wherein the internal standard is a stable isotope labeled pentasacchride.

5. The method of claim 4, wherein the stable isotope labeled pentasacchride is d₆-H3N2b.

6. The method of claim 3, wherein protein precipitation is used to deplete one or more protein(s).

7. The method of claim 3, wherein the pentasacchride are derivatized with 2-aminobenzoic acid (2-AA).

8. The method of claim 3, wherein measuring further comprises performing liquid chromatography-mass spectrometry with the sample comprising derivatized pentasaccharide and the internal standard to detect and measure the concentration of at least one pentasaccharide.

9. The method of any one of claims 1 to 8, wherein the measured pentasacchride is H3N2a and/or H3N2b.

10. The method if any one of claims 1 to 9, wherein the measured pentasacchride level is compared to a reference value of a healthy control.

11. The method of claim 10, wherein if the measured pentasacchride level is greater than the reference value the subject is classified as having GM1-gangliosidosis.

12. Use of the measured pentasacchride level of any one of claims 1 to 9 to select a therapeutic agent or a diagnostic agent for a subject.

13. The method of any one of claims 1 to 12, wherein the method further comprises administering a pharmaceutical composition to a subject classified has having GM1-gangliosidosis.

14. The method of claim 13, wherein the pharmaceutical composition comprises a substrate reduction therapy, bone marrow transplantation, pharmacological chaperone, enzyme replacement therapy, or gene therapy.

15. The method of claim 14, wherein the enzyme replacement or gene therapy is providing a functional β-galactosidase enzyme.

16. A method for treating a subject or monitoring the effectiveness of a therapeutic agent, the method comprising:
(a) providing a first biological sample obtained from a subject;
(b) administering a pharmaceutical composition to the subject;
(c) providing a second biological sample obtained from the subject sometime after administration of the pharmaceutical composition; and
(d) measuring in each sample a pentasacchride level.

17. The method of claim 16, wherein the biological sample is a urine, a blood, or a CSF sample.

18. The method of claim 17, wherein measuring pentasaccharide levels includes processing the urine, the blood sample or the CSF sample from the subject comprising the steps of adding an internal standard, depleting one or more protein(s) and derivatizing one or more pentasaccharides.

19. The method of claim 18, wherein the internal standard is a stable isotope labeled pentasacchride.

20. The method of claim 19, wherein the stable isotope labeled pentasacchride is d₆-H3N2b.

21. The method of claim 18, wherein protein precipitation is used to deplete one or more protein(s).

22. The method of claim 18, wherein the pentasacchride are derivatized with 2-aminobenzoic acid (2-AA).

23. The method of claim 18, wherein measuring further comprises performing liquid chromatography-mass spectrometry with the sample comprising derivatized pentasaccharide and the internal standard to detect and measure the concentration of at least one pentasaccharide.

24. The method of any one of claims 16 to 23, wherein the measured pentasacchride is H3N2a and/or H3N2b.

25. The method if any one of claims 1 to 9, wherein the measured pentasacchride level is compared to a reference value of a healthy control.

26. The method of claim 10, wherein if a decrease in the pentasacchride level from the first sample relative to the second sample indicates the pharmaceutical composition is effective or no change or an increase in pentasacchride level indicates the pharmaceutical composition is ineffective.

27. The method of any one of claims 16 to 26, wherein the pharmaceutical composition comprises a substrate reduction therapy, bone marrow transplantation, pharmacological chaperone, enzyme replacement therapy, or gene therapy.

28. The method of claim 27, wherein the enzyme replacement or gene therapy is providing a functional β-galactosidase enzyme.

29. The method of any one of the preceding claims, wherein the biological sample is a urine sample and the subject is classified as having GM1-gangliosidosis or treated for GM1-gangliosidosis when the measured level of H3N2b is greater than about 1.05 ng/μg creatinine.

30. The method of any one of the preceding claims, wherein the biological sample is a plasma sample and the subject is classified as having GM1-gangliosidosis or treated for GM1-gangliosidosis when the measured level of H3N2b is greater than about 8.9 ng/mL.

31. The method of any one of the preceding claims, wherein the biological sample is a CSF sample and the subject is classified as having GM1-gangliosidosis or treated for GM1-gangliosidosis when the measured level of H3N2b is greater than about 15.2 ng/mL.
